(19) United States
(12) Patent Application Publication
(10) Pub. No.: US 2026/0224867 A1
(43) Pub. Date: Aug. 6, 2026

(54) IMPLANTABLE FLUID PRESSURE AND FLOW SENSOR WITH DRIFT COMPENSATION AND METHODS OF USING THE SAME

(71) Applicant: ShuntAware Technologies, Inc., Brentwood, TN (US)

(72) Inventors: Steven Charles CICCARELLI, Brentwood, TN (US); Gabriel Michel REBEIZ, Brentwood, TN (US); Christopher Steven CICCARELLI, Brentwood, TN (US); Terry DAGLOW, Brentwood, TN (US)

(21) Appl. No.: 19/636,390

(22) Filed: Apr. 1, 2026

Related U.S. Application Data
(63) Continuation of application No. PCT/US2024/049858, filed on Oct. 3, 2024.
(60) Provisional application No. 63/587,937, filed on Oct. 4, 2023.
(60) Provisional application No. 63/631,884, filed on Apr. 9, 2024.
(60) Provisional application No. 63/679,949, filed on Aug. 6, 2024.

(51) Int. Cl. A61M 27/00

(52) U.S. Cl. CPC A61M 27/006; A61M 2205/04; A61M 2205/3334; A61M 2205/3523; A61M 2205/52; A61M 2210/0687; A61M 2230/50; A61M 2230/63

(57) ABSTRACT

Described are systems, methods, and media for monitoring intracranial pressure (ICP) in a subject.

### CROSS-REFERENCE

This application is a continuation application of International Patent Application No. PCT/US2024/049858, filed Oct. 3, 2024, which claims the benefit of U.S. Provisional Application No. 63/587,937, filed Oct. 4, 2023, U.S. Provisional Application No. 63/631,884, filed Apr. 9, 2024, and U.S. Provisional Application No. 63/679,949, filed Aug. 6, 2024, which applications are incorporated herein by reference.

### BACKGROUND

Hydrocephalus is a neurological condition in which accumulation of cerebrospinal fluid (CSF) occurs within the brain. This frequently causes increased intracranial pressure (ICP), e.g., increased pressure within the skull, in a subject, which can cause significant symptoms in both infants and older people. Hydrocephalus can be caused by a birth defect, intraventricular hemorrhage, or present later in life. The present disclosure discusses methods for monitoring, detecting changes in, and treating hydrocephalus.

### SUMMARY

Presently, a ventriculoperitoneal (VP) shunt of a subject can only be adjusted by medical personnel who may lack information of the subject's ICP. Optimizing the VP shunt opening pressure in current shunts may require multiple trips to the medical professional's office over lengthy periods of time, during which elevated or reduced CSF flows may cause a subject side effects such as headaches. Valve opening pressure optimization may be further complicated by some subjects' lack of ability to communicate headache pain to their medical professionals. An example solution for measuring ICP at present is to surgically implant a temporary pressure monitor which requires a physical wired connection from the subject to equipment in the hospital room. This implanted pressure monitor cannot be worn long-term and can only communicate with hospital equipment through a physical wired connection from the subject to the machine presenting a rather diminished quality of life for the subject to be tethered to stationary hospital equipment. Additionally, present example solutions do not account for a subject's position e.g., laying down, sitting up, standing, etc., that may affect the ICP reading and provide a false positive or false negative ICP reading leading to inaccurate follow up treatment or adjustment of VP shunt valve position. Unfortunately, when a subject's ICP pressure measurements are taken in a hospital, the subject's position is not recorded, so they must remain still to avoid corrupting the measurement further producing diminished quality of life effects and inconvenience. Generally, ICP is measured relative to atmospheric pressure; therefore, the absolute ICP is ICP plus the atmospheric pressure.

As such, the systems, platforms, and methods described herein can measure the subject's ICP, CSF flow rate, and subject position e.g., outside of a hospital environment to facilitate optimization of the VP shunt valve opening pressure threshold, and to detect shunt hardware failure, malfunction, and/or occlusion.

Aspects of the disclosure describe an implantable system for monitoring intracranial pressure (ICP) in a subject, the system comprising: a conduit for the flow of cerebrospinal fluid (CSF); a hermetically sealed compartment; a first pressure sensor configured to measure an absolute ICP and output a measurement, wherein the first pressure sensor is hermetically sealed in the compartment, and wherein the first pressure sensor comprises a surface exposed to the CSF in the conduit; a microcontroller disposed in the hermetically sealed compartment that receives multiple inputs including at least the output of the first pressure sensor; and an accelerometer disposed in the hermetically sealed compartment for monitoring a subject's positional status, and wherein the accelerometer communicates an output to the microcontroller.

Aspects of the disclosure describe an implantable system for monitoring intracranial pressure (ICP) in a subject, the system comprising: a conduit for the flow of cerebrospinal fluid (CSF); a hermetically sealed compartment; a flow rate sensor disposed in the hermetically sealed compartment, mounted to an outer surface of the CSF conduit such that the sensor is hermetically isolated from the CSF, and wherein the flow rate sensor outputs a measurement; a microcontroller disposed in the hermetically sealed compartment that receives a plurality of inputs including at least the output of the flow rate sensor; and an accelerometer disposed in the hermetically sealed compartment for monitoring a subject's positional status and to communicate an output to the microcontroller.

In some embodiments, the hermetically sealed compartment comprises a wireless charging circuit configured to be charged by an external charger. In some embodiments, the implantable system and a ventriculoperitoneal (VP) shunt valve are integrated. In some embodiments, the implantable system is proximal and connected to a VP shunt valve. In some embodiments, electronic components of the implantable system are mounted on a printed circuit board (PCB) within the hermetically sealed compartment. In some embodiments, the absolute ICP is the sum of ICP and atmospheric pressure. In some embodiments, the implantable system comprises a circuit configured to measure a common mode output voltage of the first pressure sensor, wherein the common mode output voltage is used to measure the subject's body temperature, and wherein the common mode output voltage is linearly and precisely related to temperature: T=aV+b, wherein a and b are constants. In some embodiments, a drift in pressure measurement of the first pressure sensor caused by temperature is corrected from the temperature measurement obtained from the common mode voltage of the first pressure sensor. In some embodiments, the microcontroller is configured to: prior to pressure measurements, turn on a switch to sample a common mode output voltage from the first pressure sensor; store the common mode output voltage; and turn off the switch to output differential pressure measurements. In some embodiments, the ICP=m*V_diff+b+TC*delta_Temp; TC=temp coeff, where m is a first constant, V_diff is the differential output of the first pressure sensor, b is a second constant, TC is a temperature coefficient, and delta_Temp is a change in temperature. In some embodiments, the wireless charging circuit uses near field communication (NFC). In some embodiments, the hermetically sealed compartment comprises a wireless modem configured to receive and transmit data between the microcontroller and an external device. In some embodiments, the hermetically sealed compartment comprises an antenna for receiving and transmitting wireless signals residing within the hermetically sealed compartment. In some embodiments, the drift in pressure measurement of the first pressure sensor is configured to be corrected by: storing an initial pressure of the air enclosed within the hermetically sealed compartment during assembly in non-volatile memory in the microcontroller; measuring a second pressure of the air in the hermetically sealed compartment using a second pressure sensor that comprises a membrane or surface exposed to the air within the compartment and not in contact with the CSF, wherein the second pressure sensor is configured to measure an atmospheric pressure within the compartment and output a second measurement; calculating a difference between the initial pressure and the second measurement to determine a sensor drift; and calculating a drift-corrected absolute ICP based at least in part on a difference between a first measurement ICP and the sensor drift, wherein the first measurement ICP is produced by the first pressure sensor. In some embodiments, the microcontroller is configured to receive the atmospheric pressure from an external device. In some embodiments, the microcontroller is configured to compute the ICP based at least in part on a difference between the absolute ICP and the atmospheric pressure. In some embodiments, the microcontroller is configured to compute the ICP based at least in part on a difference between the drift-corrected absolute ICP and the atmospheric pressure. In some embodiments, the external device comprises a smartphone or a tablet computer, wherein the microcontroller is configured to transmit a plurality of measurements output by an analog-to-digital converter (ADC) to the external device, and wherein the ICP is calculated by an internal processor digital signal processor (DSP) in the external device. In some embodiments, a subject status includes a message to be displayed on a graphical user interface of the external device including a diagnostic summary of ICP health, flow rate health, or recommendation for adjustment to flow resistance of the VP shunt valve, or any combination thereof. In some embodiments, the microcontroller is configured to send a message to the external device for display on the graphical user interface. In some embodiments, the external device is connected to a cloud network. In some embodiments, the CSF flowing out of the conduit is directly input to a VP shunt. In some embodiments, a subject positional status includes at least: sitting, standing, or laying down. In some embodiments, the microcontroller is configured to exclude pressure measurements if the accelerometer indicates subject movement during the measurement, wherein data is excluded upon detection of motion, and wherein data will begin to be captured again if two conditions are met: a programmable timer expires, and no motion of the subject is detected. In some embodiments, the threshold for motion detection is increased to allow for light activity. In some embodiments, a lowpass filter time constant is applied to the ICP waveform to reduce variation resulting from a motion detection threshold. In some embodiments, the microcontroller is configured to transmit a change in subject position to the external device if the accelerometer detects a change in the subject positional status from laying to sitting, or from sitting to laying, sitting to standing, laying to standing, or any combination thereof. In some embodiments, the implantable system comprises a motor. In some embodiments, the microcontroller is configured to control the motor to adjust the flow resistance of the VP shunt valve by one or more increments. In some embodiments, the microcontroller is configured to compensate for increase in CSF flow upon standing or decrease in CSF flow upon laying down, based on input from the accelerometer, by commanding a motor to adjust the flow resistance of the VP shunt valve by one or more increments. In some embodiments, the implantable system comprises a valve, wherein the valve comprises one or more of a ball-spring valve, a floating ball valve, a trunnion mounted ball valve, a top entry ball valve, a side entry ball valve, a three-way ball valve, a ball check valve, a flapper check valve, a disc check valve, a ball relief valve, a spring safety valve, or a spring-loaded pressure relief valve. In some embodiments, the implantable system comprises the controller and a shunt wireless communication device. In some embodiments, the valve, the flow rate sensor, the first pressure sensor, the shunt wireless communication device, the controller, or any combination thereof is magnetic or non-magnetic. In some embodiments, an opening pressure threshold of the valve is manually adjustable. In some embodiments, the opening pressure threshold of the valve is manually adjustable by an external magnetic device. In some embodiments, the implantable system comprises a motor configured to adjust an opening pressure threshold of the valve. In some embodiments, the controller is configured to direct the motor to adjust the opening pressure threshold. In some embodiments, the shunt wireless communication device is configured to receive a target opening pressure threshold, and wherein the controller is configured to control the motor based at least in part on the target opening pressure threshold. In some embodiments, a sensor circuit detects a motor position and the controller configures the modem to transmit this position to an external wand device. In some embodiments, the controller is configured to adjust the opening pressure threshold of the valve based at least in part on the ICP, the first measurement, the second measurement, a third measurement, or any combination thereof. In some embodiments, the controller is configured to receive a motor position from a position sensor, and wherein the shunt wireless communication device is configured to transmit an opening pressure threshold confirmation based on the motor position. In some embodiments, the implantable system comprises a memory configured to store the first measurement, second measurement, ICP, sensor drift, target ICP, or any combination thereof. In some embodiments, the memory comprises a non-volatile memory. In some embodiments, the controller is configured to adjust the opening pressure threshold of the valve based on one or more of a machine learning model, artificial intelligence, and signal processing techniques. In some embodiments, the machine learning model is configured to determine the opening pressure threshold of the valve based on the ICP, the first measurement, the second measurement, the third measurement, the CSF flow rate, or any combination thereof, and wherein the controller is configured to adjust the opening pressure threshold of the valve. In some embodiments, the status comprises one or more of a sleeping status, an awake status, an exercising status, a standing status, a sitting status, a laying status, and a circadian status. In some embodiments, an algorithm is trained on a history of ICP waveforms, flow rate waveforms, or a combination thereof, and used to determine the status of the subject, wherein Fourier analysis or other signal processing techniques are applied to the ICP waveforms or flow rate waveforms, or both, to generate training data for the algorithm. In some embodiments, the controller is configured to determine a rate of number of times the valve opens per unit of time, and wherein the rate is outputted, used to train the algorithm, or a combination thereof. In some embodiments, the first pressure sensor comprises: a capacitive sensor; a piezoelectric sensor; a piezoresistive sensor; a magnetic sensor; a resonant sensor; an optical sensor; a MEMS sensor; or any combination thereof. In some embodiments, the CSF flow rate sensor comprises: a thermal sensor; an optical sensor; an electromagnetic sensor; a Lorentz force velocity sensor; an ultrasonic sensor; or any combination thereof. In some embodiments, the implantable system comprises an energy storage device configured to power the first pressure sensor, the flow rate sensor, the controller, the accelerometer, or any combination thereof. In some embodiments, the wireless charging circuit is configured to receive power to charge the energy storage device, power the controller, or both. In some embodiments, the energy storage device comprises a battery, a capacitor, a supercapacitor, or any combination thereof. In some embodiments, the controller is configured to adjust the opening pressure threshold to a base opening pressure threshold if a charge of the energy storage device falls below a preset charge threshold. In some embodiments, the implantable system comprises a digital modulator configured to modulate a signal from the controller, and wherein the controller and the shunt wireless communication device are communicably coupled by the digital modulator. In some embodiments, the implantable device comprises a first analog to digital (A/D) converter communicably coupling the first pressure sensor and the controller and configured to convert an analog signal from the first pressure sensor to a digital signal. In some embodiments, the implantable device comprises an A/D converter communicably coupling the flow rate sensor and the controller and configured to convert an analog signal from the flow rate sensor to a digital signal. In some embodiments, the controller comprises a digital modulator, one or more A/D converters, or any combination thereof. In some embodiments, a P1 component and a P2 component of an ICP waveform are compared to determine if the subject's ICP is healthy or elevated, wherein an increased amplitude of the P2 component relative to the P1 component indicate that a subject has an elevated ICP. In some embodiments, an alert is sent to the external device when a ratio of the P2 component to the P1 component of the ICP waveform exceeds a threshold indicating that the subject has an elevated ICP. In some embodiments, an alert is sent to the external device if a difference between the P2 component and the P1 component of the ICP waveform exceeds a threshold indicating that the subject has an elevated ICP.

Aspects of the disclosure describe a platform for monitoring intracranial pressure (ICP) of cerebrospinal fluid (CSF) in a subject, the platform comprising: the implantable system; and a wand device comprising a wand wireless communication device configured to transmit the atmospheric pressure and receive the ICP from a shunt wireless communication device. In some embodiments, the first measurement includes the absolute ICP, atmospheric pressure, and sensor drift, and wherein the second measurement includes an atmospheric pressure and sensor drift, wherein the difference between the first and second measurements is the drift corrected, absolute ICP.

In some embodiments, the implantable system comprises a motor configured to adjust an opening pressure threshold of the valve, wherein the wand wireless communication device is configured to transmit a target opening pressure threshold to the implantable system, and wherein the controller is configured to receive the target opening pressure threshold and control the motor to adjust the opening pressure threshold of the valve based at least in part on the target opening pressure threshold. In some embodiments, an accelerometer is configured to output subject position data to the microcontroller, wherein upon detecting a transition between positions, the controller can direct the motor to reduce CSF flow (e.g., by increasing resistance) and in so doing, act as an electronically controlled anti-siphon device (ASD). In some embodiments, the controller is configured to adjust the opening pressure threshold of the valve based at least in part on the ICP, the first measurement, the second measurement, or any combination thereof. In some embodiments, the controller is configured to receive a motor position from a motor position sensor, and wherein the shunt wireless communication device is configured to transmit an opening pressure threshold confirmation based on the motor position. In some embodiments, the wand wireless communication device is configured to receive the opening pressure threshold confirmation. In some embodiments, the controller is configured to adjust the opening pressure threshold of the valve based on one or more of a machine learning model, artificial intelligence, or a signal processing algorithm. In some embodiments, a system algorithm is configured to determine a status of the subject based on the ICP, the first measurement, the second measurement, or any combination thereof, and wherein the controller is configured to adjust the opening pressure threshold of the valve based on the status. In some embodiments, the status comprises one or more of a sleeping status, an awake status, an exercising status, a standing status, a sitting status, a laying status, and a circadian status. In some embodiments, a system algorithm is trained on a history of ICP waveforms, flow rate waveforms, or a combination thereof, and used to determine the status of the subject, wherein Fourier analysis or other signal processing techniques are applied to the ICP waveforms, flow rate waveforms, or a combination thereof, to generate the training data for an algorithm. In some embodiments, the controller is configured to determine a valve opening rate (e.g., the number of times the valve opens per unit of time), and wherein the valve opening rate is outputted, used to train an algorithm, or a combination thereof. In some embodiments, the implantable system comprises a memory configured to store the first measurement, the second measurement, absolute ICP, ICP, the target ICP, or both. In some embodiments, the access to implantable system data, including ICP, absolute ICP, and flow rate, is restricted by a security code. In some embodiments, a status of the shunt and subject is reported via a software application residing on the wand device, wherein the status comprises information including: shunt health, mean CSF flow rate, mean ICP, or any combination thereof. In some embodiments, the memory comprises a non-volatile memory. In some embodiments, the valve comprises one or more of a ball-spring valve, a floating ball valve, a trunnion mounted ball valve, a top entry ball valve, a side entry ball valve, a three-way ball valve, a ball check valve, a flapper check valve, a disc check valve, a ball relief valve, a spring safety valve, and a spring-loaded pressure relief valve. In some embodiments, the first pressure sensor comprises: a capacitive sensor; a piezoelectric sensor; a piezoresistive sensor; a magnetic sensor; a resonant sensor; an optical sensor; a MEMS sensor; or any combination thereof. In some embodiments, the implantable system comprises an energy storage device configured to power the valve, the first pressure sensor, the second pressure sensor, the controller, or any combination thereof. In some embodiments, the platform comprises a wireless charging circuit configured to receive power to charge the energy storage device, power the controller, or a combination thereof. In some embodiments, the energy storage device comprises a battery, a capacitor, a supercapacitor, or any combination thereof. In some embodiments, the controller of the implantable system is configured to adjust the opening pressure threshold to a base opening pressure threshold if a charge of the energy storage device falls below a preset charge threshold. In some embodiments, the platform comprises a digital modulator, and wherein the controller and the shunt wireless communication device are communicably coupled by the digital modulator. In some embodiments, the platform comprises a first analog to digital (A/D) converter communicably coupling the first pressure sensor and the controller. In some embodiments, the platform comprises a second A/D converter communicably coupling the second pressure sensor and the controller. In some embodiments, the controller comprises a digital modulator, one or more A/D converters, or any combination thereof. In some embodiments, the platform comprises third A/D converter communicably coupling the flow rate sensor and the controller. In some embodiments, the wand wireless communication device is configured to transmit a target opening pressure, wherein the shunt wireless communication device is configured to receive the target opening pressure threshold, and wherein the controller is configured to direct the motor based on the target opening pressure threshold. In some embodiments, the wand device comprises a display configured to show data based on the first measurement, second measurement, the ICP, or both; the wand device comprises a speaker configured to emit a sound based on the ICP, CSF flow rate, or both; or the wand wireless communication device is configured to transmit the first measurement, second measurement, the third measurement, the ICP, CSF flow rate, or any combination thereof to a cloud storage. In some embodiments, the wand device comprises a display configured to show data based on the opening pressure threshold confirmation; the wand device comprises a speaker configured to emit a sound based on the opening pressure threshold confirmation; the wand wireless communication device is configured to transmit the opening pressure threshold confirmation to a cloud storage; or any combination thereof. In some embodiments, the wand device comprises a display configured to show data based on the target opening pressure; the wand wireless communication device is configured to transmit the target opening pressure to a cloud storage; or both. In some embodiments, the wand device, typically a smartphone running an app, is capable of uploading patient data to a private or hybrid cloud accessible by the patients' doctors, and applications in the cloud can store the patient data and process the patient data for doctors to make informed clinical diagnoses, wherein these data comprise mean ICP, mean CSF flow rate, valve opening rate, AIr, signal processing information from a system algorithm, or a combination thereof.

Aspects of the disclosure describe a computer-implemented method for monitoring intracranial pressure (ICP) of cerebrospinal fluid (CSF) in a subject, the method comprising: measuring, by an ICP sensor, an absolute ICP plus drift of the subject as a first measurement; measuring, by a second sensor, an atmospheric pressure plus drift as a second measurement; receiving the atmospheric pressure from an external source as the third measurement; calculating an absolute ICP based at least in part on a difference between the first and second measurements; calculating the ICP based at least in part on a difference between the absolute ICP and atmospheric pressure (the third measurement); and transmitting the ICP. In some embodiments, the first measurement includes the absolute ICP, atmospheric pressure, and sensor drift, and wherein the second measurement includes an atmospheric pressure and the sensor drift. In some embodiments, the third measurement is from a smartphone. In some embodiments, the computer-implemented method comprises adjusting an opening pressure of a valve. In some embodiments, the adjustment is performed manually. In some embodiments, the adjustment is performed by a magnetic device. In some embodiments, the adjustment is performed by a motor. In some embodiments, the computer-implemented method comprises receiving a target opening pressure threshold, wherein the adjustment is performed based on the target opening pressure threshold. In some embodiments, the computer-implemented method comprises storing the target opening pressure threshold to a memory. In some embodiments, the computer-implemented method comprises storing the ICP, the first measurement, the second measurement, the third measurement, or any combination thereof to a memory. In some embodiments, the adjustment is performed based at least in part on the ICP, the first measurement, the second measurement, the third measurement, or any combination thereof. In some embodiments, the adjustment is performed based on an algorithm comprising one or more of a machine learning model, artificial intelligence model, a signal processing algorithm, or any combination thereof. In some embodiments, the algorithm is configured to determine a status of the subject based on the ICP, the first measurement, the second measurement, the third measurement, or any combination thereof. In some embodiments, the status of the subject comprises one or more of a sleeping status, an awake status, an exercising status, a standing status, a sitting status, a laying status, and a circadian status. In some embodiments, a system algorithm is trained on a history of ICP waveforms and used to determine the status of the subject, wherein Fourier analysis or other signal processing techniques are applied on the ICP waveforms to generate the training data for an algorithm. In some embodiments, the controller is configured to determine a rate of number of times the valve opens per unit of time, and wherein the rate is outputted, used to train an algorithm, or a combination thereof. In some embodiments, the computer comprising adjusting the opening pressure threshold to a base opening pressure threshold if a charge of the energy storage device falls below a preset charge threshold.

Aspects of the disclosure describe a non-transitory computer-readable media comprising executable instructions that, when executed, cause at least one computer processor to perform the methods described herein.

Aspects of the disclosure describe a computer system comprising a memory storing computer-readable instructions and at least one processor configured to execute the computer-readable instructions that are configured to perform the method of any of the preceding claims.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1A illustrates example ICP waveforms of the respiratory pressure and arterial pressure cycles, as described in one or more embodiments herein.

FIG. 1B illustrates a close-up of an example ICP waveform for an unaffected subject, as described in one or more embodiments herein.

FIG. 1C illustrates a close-up of an example ICP waveform for a subject with elevated ICP, as described in one or more embodiments herein.

FIG. 2 shows a diagram of an example shunt system, as described in one or more embodiments herein.

FIG. 3 shows a diagram of an example shunt platform, as described in one or more embodiments herein.

FIG. 4 shows a diagram of an example shunt system configured to run in manual mode, as described in one or more embodiments herein.

FIG. 5 shows a diagram of an exemplary shunt system configured to run in manual mode, adjust-assist mode, or autonomous mode, as described in one or more embodiments herein.

FIG. 6 shows a diagram of an example sensor system which pairs with a shunt valve, connecting in series via catheter tubing, as described in one or more embodiments herein.

FIG. 7 shows a non-limiting example of a computing device; in this example, a device with one or more processors, memory, storage, and a network interface, as described in one or more embodiments herein.

FIG. 8 shows a diagram of an example algorithm for performing the methods described herein, as described in one or more embodiments herein.

FIG. 9 shows a diagram of an example shunt platform with cloud connectivity, as described in one or more embodiments herein.

FIG. 10 shows a diagram of an example method of monitoring ICP of CSF in a subject, as described in one or more embodiments herein.

FIG. 11 shows a diagram of an example sensor system in proximity to and/or coupled to a valve, as described in one or more embodiments herein.

FIG. 12 shows example radiological images of the brain of an individual with enlarged ventricles, due to acute shunt failure, as described in one or more embodiments herein.

FIG. 13A shows an example diagram of the implantable system, as described in one or more embodiments herein.

FIG. 13B illustrates an example exterior perspective of the implantable system, as described in one or more embodiments herein.

FIG. 14A shows an example diagram of the wireless connection between the smartphone app, the cloud, and the shunt system and/or the sensor system, as described in one or more embodiments herein.

FIG. 14B shows an example diagram of the shunt system and/or sensor system and external interfaces, as described in one or more embodiments herein.

FIG. 15 shows an example diagram illustrating the wireless connections between the cloud, the smartphone app, and the valve, as described in one or more embodiments herein.

FIG. 16 shows an example diagram of how heart rate measurements may be integrated with ICP and flow rate in the implantable system, as described in one or more embodiments herein.

FIG. 17A illustrates an example perspective view of different components within the implantable system, as described in one or more embodiments herein.

FIG. 17B illustrates an example perspective view of an exploded diagram of the components within the implantable system, as described in one or more embodiments herein.

FIG. 18 illustrates an example temperature and/or pressure switch used in the implantable system, as described in one or more embodiments herein.

FIG. 19 shows an example ICP waveform and sample data points, as described in one or more embodiments herein.

### DETAILED DESCRIPTION

Hydrocephalus is a neurological disorder in which a subject may have an imbalance in the production and absorption of CSF. Hydrocephalus can be caused by an intraventricular hemorrhage, or birth defects, such as neural tube defects. Hydrocephalus can also be acquired and/or present later in life, such as from meningitis, brain tumors, traumatic brain injury, intraventricular hemorrhage, or subarachnoid hemorrhage. As many as 1 in 1,000 people may be born with hydrocephalus. Hydrocephalus is characterized by impaired/obstructed CSF flow, impaired CSF reabsorption, or excessive CSF production. Accumulation of CSF within the brain can cause increased intracranial pressure, which can lead to various symptoms in both infants and the elderly. Infants may show a rapid increase in head size. The elderly may have headaches, double vision, poor balance, urinary incontinence, personality changes, or mental impairment.

Surgical treatment may frequently be used to treat subjects with hydrocephalus, as the accumulation of CSF can crush the brain against the skull, leading to death if not treated. Long-term treatment may frequently include an implant and adjustment of a cerebral shunt. Creating a shunt may involve the placement of a ventricular catheter into the cerebral ventricles to drain the excess CSF into other body cavities, from where it can be reabsorbed. Frequently, a shunt may drain the CSF into the peritoneal cavity (e.g., a ventriculoperitoneal shunt). A ventriculoperitoneal shunt may comprise a proximal catheter, a valve, and a distal catheter.

Although shunts may be highly beneficial for subjects with hydrocephalus, there can be significant complications or difficulties with commonly-used shunts. For example, shunts frequently may only be adjusted through surgery or other in-office adjustment by a skilled physician or medical professional at a e.g., a hospital. Additionally, shunts may not be able to adjust to CSF changes resulting from body mass fluctuations of the subject, especially during growth spurts, puberty, elevation changes, or long-term ventricle changes. Further, titrating the CSF flow in shunts may require multiple trips to the medical professional's office over lengthy periods of time. During this titration period, elevated or reduced CSF flows may cause side effects such as headaches. CSF flow optimization may also be further complicated by some subjects' lack of ability to communicate headache pain to their medical professionals, such as in young children. Additionally, the magnetic components in many shunts may present a risk of causing valve opening pressure adjustment when receiving a magnetic resonance imaging (MRI) scan.

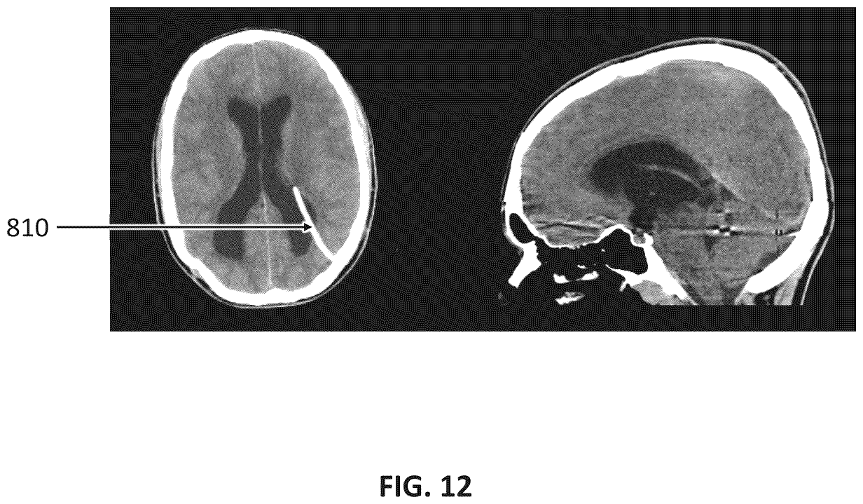

Further, shunts that employ valves with a fixed opening pressure threshold may not be able to adapt to their subject and CSF pressures. Such simple valves may cause headaches or neurological side effects due to sub-optimal CSF flow, resulting in elevated ICP or low ICP. While adjustable valves may allow opening pressure threshold adjustment by a medical professional during an office visit, the exact opening pressure threshold setting may be difficult to confirm without radiological imaging. Symptoms of a clogged or infected shunt may include vomiting, lethargy, fever, and/or headache, which may ultimately lead to coma or death. Avoiding such outcomes is thus a high priority in the treatment of hydrocephalus. FIG. 12 shows an example radiological images of the brain of an subject with acute shunt failure. Despite the placement of the proximal catheter 810 into the individual's brain, it can be seen that the individual's ventricles are enlarged, as they are filled with excess CSF that is not properly flowing through the shunt system.

As such, the systems, platforms, and methods described herein can measure the subject's ICP to provide data to assist in optimizing the opening pressure threshold, as well as detecting shunt hardware failure or occlusion. The systems and/or methods described herein may couple to an existing VP shunt valve to record shunt information (e.g., diagnostic information) and communicate (e.g., over a wireless communication) such diagnostic information to a smartphone or other computing device for further monitoring and/or processing of the diagnostic information to improve the performance of the existing VP shunt. This may allow a subject to avoid needless trips to the emergency room or other healthcare provider.

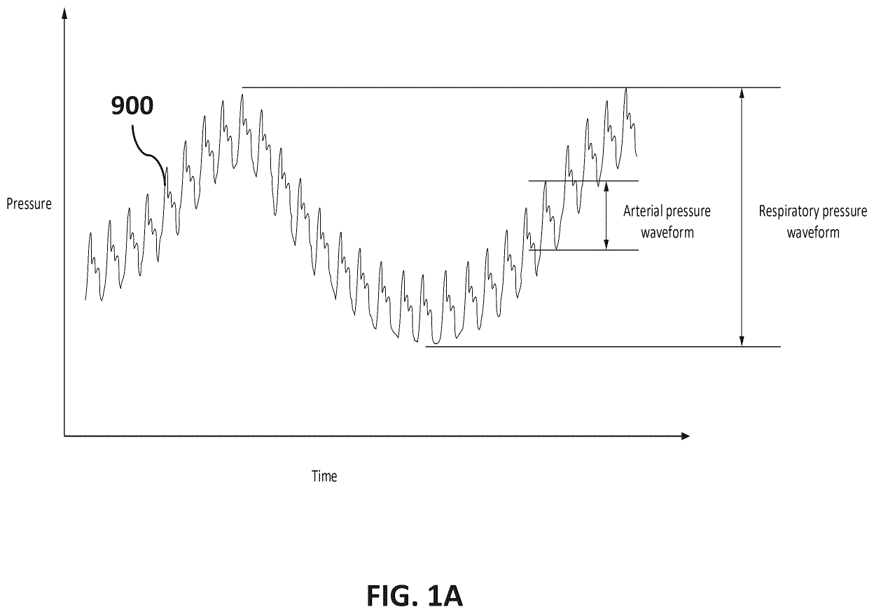

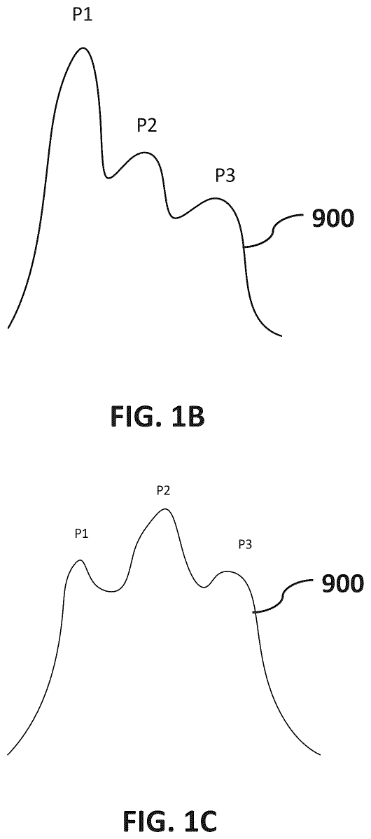

FIGS. 1A-1C show an example ICP waveform comprising respiratory and arterial cycles, in accordance with some embodiments. The mean ICP may be calculated as the time average of the waveform in FIG. 1A. An ICP waveform 900 may comprise a respiratory cycle, an arterial cycle, and Lundberg A and B waves. In subjects with elevated ICP resulting from excess CSF, variations in the arterial cycle may be seen. FIG. 1B shows a close-up of an example ICP waveform for an unaffected subject with a normal arterial cycle. Each wave in the ICP waveform may have P1, P2, and/or P3 components as shown in FIGS. 1B-1C.

The arterial cycle may comprise P1, P2, and P3 waves. P1 waves (percussion/systolic waves) may be produced by systolic pressure transmitted to the choroid plexus. P1 waves may be generated by a mechanism by which CSF is produced. P2 waves (elastance/tidal waves) may be produced by the restriction of ventricular expansion by the rigid dura and scull, in a similar manner to an echo. P3 waves (dicrotic waves) may be produced by closure of the aortic valve. As shown in FIG. 1C, when ICP is elevated, the P2 wave may increase in amplitude, altering the shape of the overall ICP waveform and its mean value.

ICP waveforms can be measured in a subject with a shunt to determine if the shunt is treating increases and/or decreases in a subject's ICP effectively. ICP for a patient with a properly functioning VP shunt may exhibit a waveform with a P2 wave similar to FIG. 1B when the patient is at rest and stationary. This can be characterized upon surgical implantation of the valve comprising sensors described herein. Each person is different; therefore, the baseline may be tailored to the individual under the direction and care of a neurosurgical team. Once a baseline ICP waveform has been characterized, it can serve as a reference for future ICP waveform measurements. The system can make a clinical recommendation based on mean ICP, the ratio of P2 to P1, the difference between P2 and P1, mean flow rate, or any combination thereof.

In an aspect, the present disclosure provides an implantable system for monitoring ICP in a subject. In some embodiments, the implantable system may comprise a conduit for the flow of CSF. In some embodiments, the implantable system may further comprise a hermetically sealed compartment. In some embodiments, the implantable system may further comprise a first pressure sensor configured to measure an absolute ICP and output a measurement, wherein the first pressure sensor may be hermetically sealed inside the compartment and may have a membrane and/or surface exposed to the CSF in the conduit. In some embodiments, the implantable system may further comprise a microcontroller disposed inside the compartment and configured to receive multiple inputs, including at least the output of the first pressure sensor. In some embodiments, the implantable system may further comprise an accelerometer disposed inside the hermetically sealed compartment for monitoring a subject positional status, which may be configured to communicate an output to a microcontroller. In some cases, the microcontroller may use or discard ICP values based on the subject's movement as measured by the accelerometer. For example, if the accelerometer determined that the subject was running, these ICP values may be discarded since the ICP values would fluctuate and vary based on the changing movement and/or motion of the subject. The accelerometer may detect whether a subject is sitting, lying down, standing up, walking, running, among other possible movements and/or positions of a subject. Problems in receiving, determining, and/or obtaining a stable ICP measurement may also occur if the subject changes position from laying to sitting or vice versa. For example, gravity can speed CSF drainage such that laying down can incur higher ICP.

In some embodiments, the implantable system may comprise a sensor system 6001 and/or shunt system (6000, 7000), described elsewhere herein.

In some cases, the microcontroller may be configured to exclude pressure measurements if the accelerometer indicates an increase in subject movement during the measurement. Data may be excluded upon detection of motion. Data may begin to be captured again if two conditions are met: (a) a programmable timer expires (e.g., a time of 3 seconds but can be more or less), and (b) there are no more motions of the subject detected. When motion occurs, data collection may be gated to omit any corrupted data. When the subject's position changes, each pressure data point may be marked with the subject's position, e.g., determined by an accelerometer and/or gyroscope. When the subject moves during measurements, the smartphone or other computing device may emit a sound and display text instructing the subject to remain still.

In some cases, the threshold for motion detection may be increased to allow for light activity of the subject. In some cases, a longer lowpass filter time constant may be applied to the ICP waveform to reduce variation resulting from the higher motion detection threshold. In some cases, the microcontroller may be further configured to transmit a change in subject position to the external device if the accelerometer detects a change in the subject positional status from laying to sitting, or from sitting to laying, sitting to standing, laying to standing, or any combination thereof. Measuring pressure may be optimal when the user remains still and in one of three positions: sitting, standing, and laying down. The user can provide their initial position to the external wand device, e.g., an app running on a smartphone, prior to measurement. The accelerometer may detect a change in the user's position relative to the initial position.

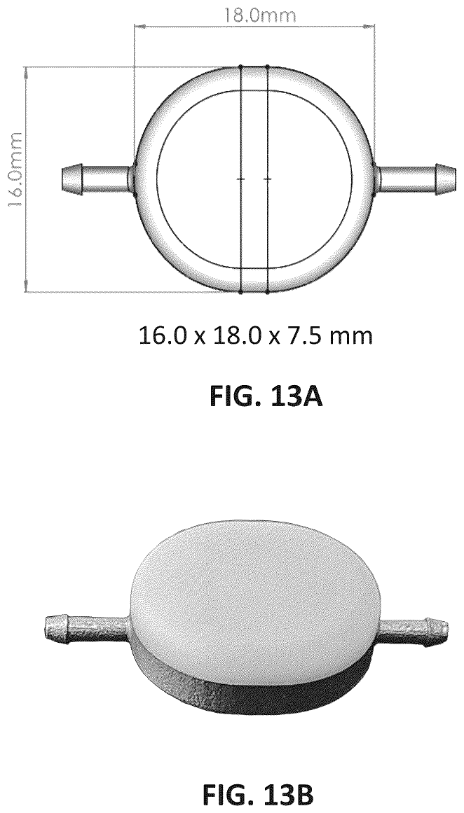

FIGS. 13A-13B illustrate an embodiment of the implantable system described herein. FIG. 13A shows an example diagram of the implantable system. FIG. 13B illustrates an exterior perspective view of the implantable system. The dimensions of the implantable system may be about 16.0 mm by about 18.0 mm by about 7.5 mm. The implantable system may have any other dimensions appropriate for such a system. The implantable system may be about the size of a U.S. penny.

In some embodiments, the implantable system may further comprise a flow rate sensor provided inside a compartment, mounted to the outside of the conduit such that the sensor is hermetically isolated from the CSF. In some embodiments, the flow rate sensor may output a CSF flow rate measurement. This flow rate sensor may be used to measure the flow rate of the CSF in a subject. Both the ICP and the flow rate can be useful in monitoring the shunt system. In some embodiments, the compartment may comprise a wireless charging circuit configured to be charged by an external charger.

In some embodiments, the implantable system and a ventriculoperitoneal (VP) shunt valve may be integrated. In some embodiments, the implantable system may be proximal and coupled to a VP shunt valve. In some embodiments, the electronic components may be mounted on a printed circuit board (PCB) within the hermetically sealed compartment.

In some cases, the absolute ICP may be defined as the sum of ICP and atmospheric pressure. In some cases, the implantable system may further comprise a circuit configured to measure a common mode output voltage of the first pressure sensor, wherein the common mode output voltage may be used to measure the subject's body temperature. The common mode output voltage may be linearly and precisely related to temperature: T=aV+b, wherein a and b are constants. This temperature measurement derived from the common mode output voltage may be used to correct a drift in pressure measurement of the first pressure sensor caused by temperature. In some embodiments, the first pressure sensor within the implantable system can obtain the body temperature from the CSF passing over the membrane and/or surface of the pressure sensor. The linear relationship between temperature and common mode output voltage can be used to compute temperature. This temperature can be transmitted to the smartphone app and incorporated into user status.

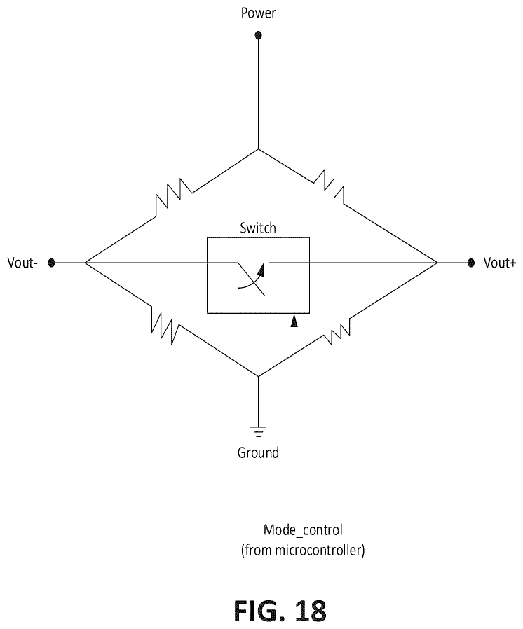

FIG. 18 illustrates a temperature and/or pressure switch that may be used in the implantable system described herein. In some cases, temperature and/or pressure switch may be in the form of a Wheatstone bridge circuit. When the switch is open, the circuit may measure pressure. When the switch is closed, the circuit may measure temperature. When the switch is closed, the system may follow the equation Vₒᵤₜ=V_{cm}, where V_{cm} is the common mode voltage. For example, the temperature may follow the equation T=a*V_{cm}+b. The constants “a” and “b” may be set during calibration and thus are shown here as examples only. The constants “a” and “b” may also be determined through characterization, e.g., characterizing one or more implantable systems and setting, identifying, and/or determining “a” and “b” for each of the one or more implantable systems. In some cases, the “a” and “b” constants may vary between the one or more implantable systems.

In some embodiments, the microcontroller may be further configured to: (a) prior to pressure measurements, turn on a switch to sample a common mode output voltage from the first pressure sensor; (b) store the common mode voltage; and (c) turn off the switch to output differential pressure measurements.

In some embodiments, a switch may be used to alternate between coupling the differential and common mode voltage outputs to the A/D. Pressure may be obtained from the differential output and body temperature may be obtained from the common mode output. These may not be simultaneously measured, so a switch controlled by the controller may determine which is being measured.

In some cases, the ICP may be defined as: ICP=m*V_diff+b-TC*delta_Temp-atmospheric pressure; TC=temp coeff, wherein m may be a first constant, V_diff may be the differential output of the first pressure sensor, b may be a second constant, TC may be a temperature coefficient, and delta_Temp may be a change in temperature from where “m” and “b” were determined. The first and second constants “m” and “b” may vary based on calibration of one or more implantable systems. Constants “m” and “b” may be characterized or calibrated at one or more temperature(s). However, “b” may change when temperature changes. This change may be included with the temperature coefficient, TC. Atmospheric pressure may comprise about 650 mmHg to about 800 mmHg. In some cases, variables TC, “m”, “b”, atmospheric pressure, or any combination thereof, may be multiplied by an amplification and/or a gain value. In some cases, the ICP determined by equations, algorithms, systems, and/or methods described elsewhere herein, may be provided to a gain processing module with offset nulling.

In some cases, the wireless charging circuit may use near field communication. In some cases, the hermetically sealed compartment may further comprise a wireless modem configured to receive and transmit data between the microcontroller and an external device, such as a tablet or smartphone. In some cases, the compartment may further comprise an antenna for receiving and transmitting wireless signals residing within the hermetically sealed compartment.

In some cases, the drift of the first pressure sensor may be configured to be corrected by: (a) storing an initial pressure of the air enclosed within the compartment during assembly in non-volatile memory in the microcontroller; (b) measuring a second pressure of the air in the compartment using a second pressure sensor that comprises a membrane and/or surface exposed to the air within the compartment and not in contact with the CSF, wherein the second pressure sensor may be configured to measure an atmospheric pressure within the compartment and output a second measurement; (c) calculating a difference between the initial pressure and the second measurement to determine a sensor drift; and (d) calculating the drift-corrected absolute ICP based at least in part on a difference between the first measurement ICP and the drift. The first measurement ICP may be produced by the first pressure sensor. Using a second pressure sensor in the implantable system described herein may allow for the drift of the first pressure sensor to be corrected.

In some cases, the microcontroller may be further configured to receive the atmospheric pressure from an external device. In some cases, the microcontroller may be further configured to compute the ICP based at least in part on a difference between the absolute ICP and the atmospheric pressure. In some cases, the microcontroller may be configured to compute the ICP based at least in part on a difference between the drift-corrected absolute ICP and the atmospheric pressure.

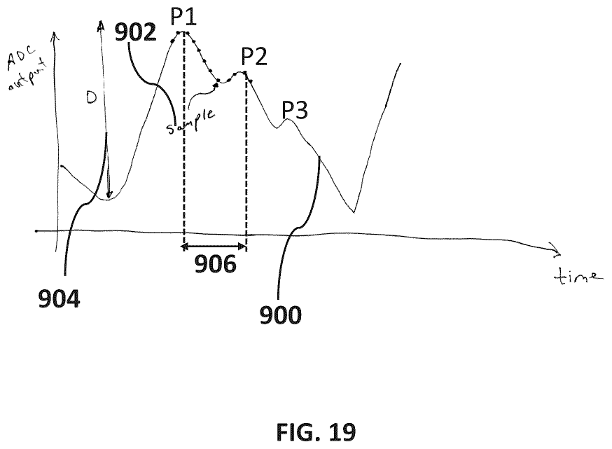

In some cases, the external device may comprise a smartphone or a tablet computer. In some cases, the external device may be a laptop, desktop computer, or other computing device. The microcontroller may be further configured to transmit a plurality of measurements output by an analog-to-digital converter (ADC) to the external device. The ICP may be calculated by an internal digital signal processor in the external device. The ADC sample rate may be set to satisfy a Nyquist sampling frequency of the highest frequency of the ICP waveform 900, described elsewhere herein, e.g., to sufficiently sample the local peaks and/or troughs of the ICP waveform as shown in FIGS. 1A-1C. In some cases, the ADC may sample the ICP waveform 900 at a rate equal to the fundamental frequency of the ICP waveform (fₛ) multiplied by the period or time between the P1 and P2 components of the ICP waveform (Δt) 906, as shown, in FIG. 19. In some cases, the P1 component of the ICP waveform may be identified and/or determined from identifying the peak of the ICP waveform 900. In some cases, the P2 component of the ICP waveform may be identified and/or determined within at least about 10 data sample points 902 from the peak P1 data sample point of the ICP waveform. In some cases, an ICP pressure labeled “D” 904 in FIG. 19, may be identified and/or determined as the amplitude of the P1 component of the ICP waveform. In some cases, the amplitude may comprise an amplitude between a trough of the ICP waveform and the peak P1 component of the ICP waveform. In some cases, “D” may comprise a value of at least about 8 mmHg. In some cases, “D” may comprise a value of up to about 20 mmHg. In some cases, if “D” comprises a value of at least about 3 mmHg, and if the peak value of P1 subtracted from the peak value of P2 is at least about 5 mmHg, then the ICP waveform of the subject may be characterized, classified, identified, and/or determined to be an ICP waveform of a healthy subject. In some cases, if “D” comprises a value of at least about 3 mmHg, and if the peak value of P1 subtracted from the peak value of P2 is up to about 5 mmHg, then the ICP waveform of the subject may be characterized, classified, identified, and/or determined to be an ICP waveform of a unhealthy subject. In some cases, if “D” comprises a value of up to about 3 mmHg, then the ICP waveform may be characterized as a non-human ICP waveform. In some cases, the non-human may comprise an animal, e.g., animals tested in a lab. In some instances, the value of “D” may be utilized and/or processed by the one or more predictive models, described herein, to classify and/or characterize whether the implantable system is implanted in a human or an animal.

In some cases, the subject status may include a message to be displayed on the graphical user interface of the external device including a diagnostic summary of ICP health, flow rate health, and/or recommendation for adjustment to flow resistance of the VP shunt valve, or any combination thereof. In some cases, the microcontroller may be further configured to send a message to the external device for display on the graphical user interface. In some cases, the external device may be connected to a cloud network. In some cases, the status may comprise one or more of a sleeping status, an awake status, an exercising status, a standing status, a sitting status, a laying status, and a circadian status.

In some cases, the CSF flowing out of the conduit may be coupled to an input of a VP shunt. In some cases, the implantable system may further comprise a motor. In some cases, the microcontroller may be further configured to control the motor to adjust the VP shunt valve resistance in one or more increments. In some cases, the microcontroller may be further configured to compensate for increases in CSF flow upon standing or decreases in CSF flow upon laying down, based on input from the accelerometer. This may be accomplished by commanding a motor to adjust the VP shunt valve resistance in one or more increments.

In some cases, the implantable system may further comprise a valve. The valve may comprise one or more of a ball-spring valve, a floating ball valve, a trunnion mounted ball valve, a top entry ball valve, a side entry ball valve, a three-way ball valve, a ball check valve, a flapper check valve, a disc check valve, a ball relief valve, a spring safety valve, or a spring-loaded pressure relief valve. In some cases, the implantable system may further comprise the controller and a shunt wireless communication device. In some cases, the valve, the flow rate sensor, first pressure sensor, the shunt wireless communication device, the controller, or any combination thereof may be magnetic or non-magnetic. In some cases, an opening pressure threshold of the valve may be manually adjustable. In some cases, the opening pressure threshold of the valve may be manually adjustable by an external magnetic device. In some cases, the implantable system may further comprise a motor configured to adjust an opening pressure threshold of the valve. In some cases, the controller may be further configured to direct the motor to adjust the opening pressure threshold. In some cases, the shunt wireless communication device may be further configured to receive a target opening pressure threshold. In some cases, the controller may be further configured to control the motor based at least in part on the target opening pressure threshold. In some cases, a sensor circuit may detect the motor position. The controller may configure the modem to transmit this position to an external device (e.g., an external wand device). In some cases, the controller may be further configured to adjust the opening pressure threshold of the valve based at least in part on the ICP, the first measurement, the second measurement, a third measurement, or any combination thereof. In some cases, the controller may be further configured to receive a motor position from a position sensor. The shunt wireless communication device may be further configured to transmit an opening pressure threshold confirmation based on the motor position.

In some cases, the implantable system may further comprise a memory configured to store the first measurement, second measurement, ICP, sensor drift, target ICP, or any combination thereof. In some cases, the memory may comprise a non-volatile memory. In some cases, the controller may be further configured to adjust the opening pressure threshold of the valve based on one or more of a machine learning model, artificial intelligence, and signal processing techniques. In some cases, a machine learning model may be configured to determine the opening pressure threshold of the valve based on the ICP, the first measurement, the second measurement, CSF flow rate, or any combination thereof, and the controller may be configured to adjust the opening pressure threshold of the valve. In some cases, a machine learning model may be configured to predict shunt failure, optimize valve flow rate, determine Slit Ventricle Syndrome (SLS) prognosis factors and/or considerations, and/or determine the source of headaches for subjects with hydrocephalus. Parameters for such a model may include, but are not limited to, ICP, CSF flow rate, user status, barometric pressure, age, years since first shunt, altitude, and headache pain. This machine learning model may be used for research insights and/or medical interventions.

In some cases, an algorithm may be trained on a history of ICP and/or flow rate waveforms and used to determine the status of the subject. Fourier analysis or other signal processing techniques may be applied to the ICP waveforms or flow rate waveforms, or both, to generate the training data for the algorithm. In some cases, the controller may be configured to determine a rate of the number of times the valve opens per unit of time. This rate may be output and/or used to train the algorithm.

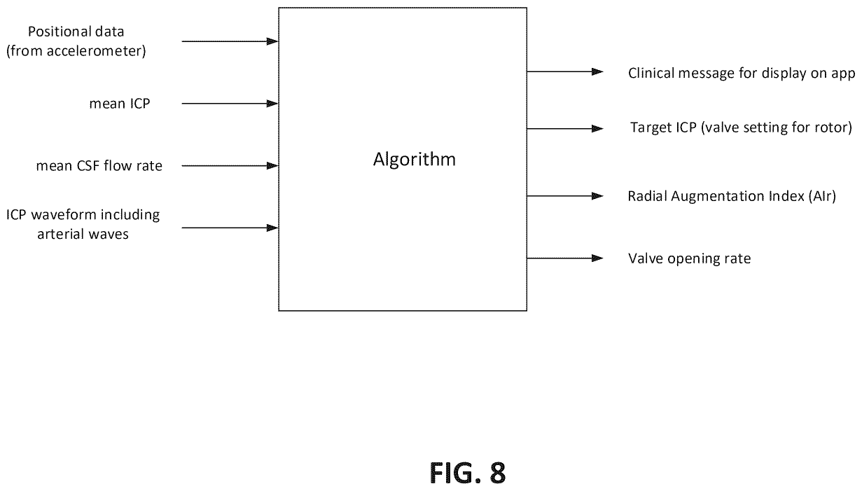

FIG. 8 shows an exemplary embodiment of an algorithm that may be used in the methods and systems described herein. Inputs to the algorithm may comprise positional data from an accelerometer, a mean ICP, a mean CSF flow rate, and/or relative harmonic levels, among other possible inputs. The algorithm may output a clinical message for display on the app of the external device and/or a target ICP/valve setting for the rotor.

As described elsewhere herein, a relationship between the P2 and P1 wave amplitudes in the ICP waveform may determine whether the subject has a healthy or elevated ICP. This relationship may be represented by the Radial Augmentation Index (AIr), which may be defined as AIr=20*log (P2/P1). These P2 and P1 amplitudes may be calculated from the relative peaks in the time domain.

Table 1 summarizes exemplary different ranges of AIr values and whether they correspond to healthy or elevated ICP. The app may display different messages based on whether the ICP is healthy or elevated based on the AIr value. These AIr thresholds may be programmable as exact thresholds may be unknown. The AIr may also correspond to P2−P1 as opposed to P2/P1.

TABLE 1
Exemplary normal and elevated AIr values.

| Status | AIr (dB) | fundamental frequency (Hz) | App Display |
|---|---|---|---|
| Normal | <−3 | 0.5-3.0 | “normal ICP” |
| | | | Display ICP average value |
| Elevated ICP | ≥−3 | 0.5-3.0 | “elevated ICP” |
| | | | Display ICP average value |
| Dangerous ICP | >+3 | 0.5-3.0 | “ICP is dangerously high” |
| | | | Display ICP average value |
| Non-human ICP | 0 | >5 Hz OR not periodic | “non-biologic detected” |
| (demo case) | | | Display average pressure value |

In some embodiments, a P1 component and a P2 component of an ICP waveform may be compared in order to determine if the subject's ICP is healthy or elevated. An increased amplitude of the P2 component relative to the P1 component may indicate that a subject has an elevated ICP. In some cases, an alert may be sent to the external device if a ratio of the P2 component to the P1 component of the ICP waveform exceeds a threshold indicating that the subject has an elevated ICP. In some cases, an alert may be sent to the external device if a difference between the P2 component and the P1 component of the ICP waveform exceeds a threshold indicating that the subject has an elevated ICP.

In some cases, the first pressure sensor may comprise: (a) a capacitive sensor; (b) a piezoelectric sensor; (c) a piezo resistive sensor; (d) a magnetic sensor; (e) a resonant sensor; (f) an optical sensor; (g) a MEMS sensor; or (h) any combination thereof. In some cases, the CSF flow rate sensor may comprise: (a) a thermal sensor; (b) an optical sensor; (c) an electromagnetic sensor; (d) a Lorentz force velocity sensor; (e) an ultrasonic sensor; or (f) any combination thereof. In some cases, the implantable system may further comprise an energy storage device configured to power the first pressure sensor, the flow rate sensor, the controller, the accelerometer, or any combination thereof. In some cases, the implantable system may further comprise a wireless charging circuit configured to receive power to charge the energy storage device, power the controller, or both. In some cases, the energy storage device may comprise a battery, a capacitor, a supercapacitor, or any combination thereof. In some cases, the controller may be further configured to adjust the opening pressure threshold to a base opening pressure threshold if a charge of the energy storage device falls below a preset charge threshold.

In some cases, the implantable system may further comprise a digital modulator configured to modulate a signal from the controller. The controller and the shunt wireless communication device may be communicably coupled by the digital modulator. In some cases, the implantable system may further comprise a first analog to digital (A/D) converter communicably coupling the first pressure sensor and the controller and configured to convert an analog signal from the first pressure sensor to a digital signal. In some cases, the implantable system may further comprise an A/D converter communicably coupling the flow rate sensor and the controller and configured to convert an analog signal from the flow rate sensor to a digital signal. In some cases, the controller may comprise a digital modulator, one or more A/D converters, or any combination thereof.

In some embodiments, the implantable system may further comprise a first analog to digital (A/D) converter communicably coupling the first pressure sensor and the controller. The first A/D converter may be configured to convert an analog signal from the sensor to a digital signal.

In some embodiments, the implantable system may further comprise a second analog to digital (A/D) converter communicably coupling the second pressure sensor and the controller. The second A/D converter may be configured to convert an analog signal from the sensor to a digital signal.

In some embodiments, the implantable system may further comprise a third A/D converter communicably coupling the flow rate sensor and the controller. The third A/D converter may be configured to convert an analog signal from the flow rate sensor to a digital signal.

In some embodiments, the implantable system may further comprise a fourth A/D converter communicably coupling the accelerometer and the controller and configured to convert an analog signal from the accelerometer to a digital signal.

In an aspect, the present disclosure provides a platform for monitoring the ICP of the CSF in a subject. The platform may comprise the implantable system described elsewhere herein and a wand device comprising a wand wireless communication device configured to transmit the atmospheric pressure and receive the ICP from the shunt wireless communication device. In some embodiments, the first measurement may include the absolute ICP, atmospheric pressure, and sensor drift. The second measurement may include an atmospheric pressure and sensor drift, wherein the difference between the first and second measurements may be the drift corrected, absolute ICP.

In some cases, the platform may further comprise a motor configured to adjust an opening pressure threshold of the valve. In some cases, the wand wireless communication device may be further configured to transmit a target opening pressure threshold to the implantable system. The controller may be further configured to receive the target opening pressure threshold and control the motor to adjust the opening pressure threshold of the valve based at least in part on the target opening pressure threshold.

In some cases, an accelerometer may be configured to output subject position data to the microcontroller. Upon detecting a transition between positions, the controller may direct the motor to reduce CSF flow (e.g., by increasing resistance), and in so doing, act as electronically controlled anti-siphon device (ASD). In some cases, the controller may be further configured to adjust the opening pressure threshold of the valve based at least in part on the ICP, the first measurement, the second measurement, or any combination thereof. In some embodiments, the controller may be further configured to receive a motor position from a motor position sensor. The shunt wireless communication device may be further configured to transmit an opening pressure threshold confirmation based on the motor position.

In some embodiments, the wand wireless communication device may be further configured to receive the opening pressure threshold confirmation. In some cases, the controller may be further configured to adjust the opening pressure threshold of the valve based on one or more of a machine learning model, artificial intelligence, or a signal processing algorithm. In some cases, a system algorithm may be configured to determine a status of the subject based on the ICP, the first measurement, the second measurement, or any combination thereof. The controller may be configured to adjust the opening pressure threshold of the valve based on the status. The status may comprise one or more of a sleeping status, an awake status, an exercising status, a standing status, a sitting status, a laying status, and a circadian status. In some cases, a system algorithm may be trained on a history of ICP and/or flow rate waveforms and used to determine the status of the subject. In some cases, Fourier analysis or other signal processing techniques may be applied to the ICP and/or flow rate waveforms to generate the training data for an algorithm.

In some cases, the controller may be configured to determine a valve opening rate (the number of times the valve opens per unit of time). The valve opening rate may be output and/or used to train an algorithm.

In some cases, the platform may further comprise a memory configured to store the first measurement, the second measurement, absolute ICP, ICP, the target ICP, or both. In some cases, access to implantable system data, including ICP, absolute ICP, and flow rate, may be restricted by a security code. In some cases, wherein a status of the shunt and subject may be reported via a software application residing on the wand device. The status may comprise information including: shunt health, mean CSF flow rate, and/or mean ICP. In some cases, the memory may comprise a non-volatile memory.

In some cases, the valve may comprise one or more of a ball-spring valve, a floating ball valve, a trunnion mounted ball valve, a top entry ball valve, a side entry ball valve, a three-way ball valve, a ball check valve, a flapper check valve, a disc check valve, a ball relief valve, a spring safety valve, and a spring-loaded pressure relief valve. In some cases, the first pressure sensor may comprise: a capacitive sensor; a piezoelectric sensor; a piezoresistive sensor; a magnetic sensor; a resonant sensor; an optical sensor; a MEMS sensor; or any combination thereof.

In some cases, the platform may further comprise an energy storage device configured to power the valve, the first pressure sensor, the second pressure sensor, the controller, or any combination thereof. In some cases, the platform may further comprise a wireless charging circuit configured to receive power to charge the energy storage device, and/or power the controller, etc., or both. In some cases, the energy storage device may comprise a battery, a capacitor, a supercapacitor, or any combination thereof. In some cases, the controller of the implantable system may be configured to adjust the opening pressure threshold to a base opening pressure threshold if a charge of the energy storage device falls below a preset charge threshold.

In some cases, the platform may further comprise a digital modulator. The controller and the shunt wireless communication device may be communicably coupled by the digital modulator. In some cases, the platform may further comprise a first analog to digital (A/D) converter communicably coupling the first pressure sensor and the controller. In some cases, the platform may further comprise a second A/D converter communicably coupling the second pressure sensor and the controller. In some cases, the controller may comprise a digital modulator, one or more A/D converters, or any combination thereof. In some cases, the platform may further comprise a third A/D converter communicably coupling the flow rate sensor and the controller.

In some cases, the wand wireless device may be further configured to transmit a target opening pressure. The shunt wireless communication device may be further configured to receive the target opening pressure threshold. The controller may be configured to direct the motor based on the target opening pressure threshold.

In some cases, the wand device may further comprise a display configured to show data based on the first measurement, second measurement, the ICP, or both. The wand device may further comprise a speaker configured to emit a sound based on the ICP, CSF flow rate, or both. The wand wireless communication device may be further configured to transmit the first measurement, second measurement, the third measurement, the ICP, CSF flow rate, or any combination thereof to a cloud storage. In some cases, the wand device may further comprise a display configured to show data based on the opening pressure threshold confirmation. The wand device may further comprise a speaker configured to emit a sound based on the opening pressure threshold confirmation. The wand wireless communication device may be further configured to transmit the opening pressure threshold confirmation to a cloud storage. In some embodiments, the wand device may further comprise a display configured to show data based on the target opening pressure. The wand wireless communication device may also be further configured to transmit the target opening pressure to a cloud storage.

In some embodiments, the wand device may be capable of uploading patient data to a private or hybrid cloud accessible by the patients' doctors. The wand device may be a smartphone or tablet running an app. The wand device may also be a laptop or other computing device. Applications in the cloud may store this patient data and further process the patient data for doctors to make informed clinical diagnoses. This patient data may include mean ICP, mean CSF flow rate, valve opening rate, and/or signal processing information from a system algorithm.

In an aspect, the present disclosure provides a computer-implemented method for monitoring ICP of CSF in a subject. The method may comprise measuring, by an ICP sensor, an absolute ICP plus drift of the subject as a first measurement; measuring, by a second sensor, an atmospheric pressure plus drift as a second measurement; receiving the atmospheric pressure from an external source as the third measurement; calculating an absolute ICP based at least in part on a difference between the first and second measurements; calculating the ICP based at least in part on a difference between the absolute ICP and atmospheric pressure (the third measurement); and transmitting the ICP.

In some embodiments, the first measurement may include the absolute ICP, atmospheric pressure, and sensor drift. The second measurement may include an atmospheric pressure and the sensor drift. In some cases, the third measurement may be from a smartphone.

In some embodiments, the computer-implemented method may further comprise adjusting an opening pressure of a valve. In some cases, the adjustment may be performed manually. In some cases, the adjustment may be performed by a magnetic device. In some cases, the adjustment may be performed by a motor. In some cases, the computer-implemented method may further comprise receiving a target opening pressure threshold. The adjustment may be performed based on the target opening pressure threshold. In some cases, the computer-implemented method may further comprise storing the target opening pressure threshold to a memory. In some cases, the computer-implemented method may further comprise storing the ICP, the first measurement, the second measurement, the third measurement, or any combination thereof to a memory. In some cases, the adjustment may be performed based at least in part on the ICP, the first measurement, the second measurement, the third measurement, or any combination thereof. In some cases, the adjustment may be performed based on an algorithm comprising one or more of a machine learning model, artificial intelligence model, a signal processing algorithm, etc. In some cases, the algorithm may be configured to determine a status of the subject based on the ICP, the first measurement, the second measurement, the third measurement, or any combination thereof. In some cases, the status of the subject may comprise one or more of a sleeping status, an awake status, an exercising status, a standing status, a sitting status, a laying status, and a circadian status. In some cases, a system algorithm may be trained on a history of ICP waveforms and used to determine the status of the subject. Fourier analysis or other signal processing techniques may be applied to the ICP waveforms to generate the training data for an algorithm.

In some embodiments, the controller may be configured to determine a rate of number of times the valve opens per unit of time. The rate may be output and/or used to train an algorithm.

In some embodiments, the computer-implemented method may further comprise adjusting the opening pressure threshold to a base opening pressure threshold if a charge of the energy storage device falls below a preset charge threshold.

In some embodiments, non-transitory computer-readable media comprising executable instructions may, when executed, cause at least one computer processor to perform the methods described elsewhere herein. In some embodiments, a computer system comprising a memory storing computer-readable instructions and at least one processor configured to execute the computer-readable instructions may be configured to perform the methods described elsewhere herein.

### Implantable System

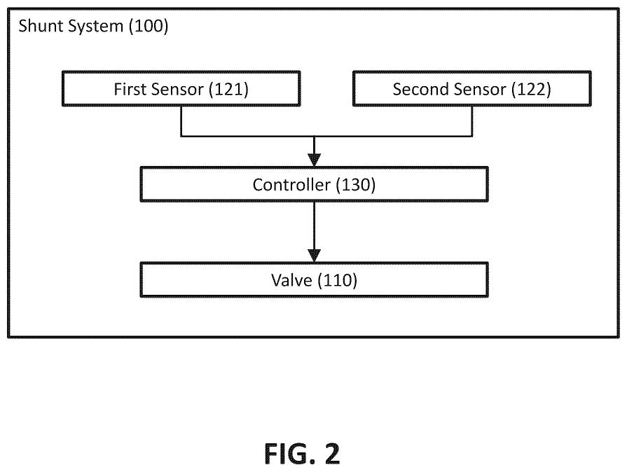

FIG. 2 shows the implantable system 100 for monitoring ICP in a subject (e.g., shunt system), in accordance with some embodiments. In some embodiments, the shunt system 100 comprises a valve 110, a ICP sensor 121, a controller 130 (e.g., a microcontroller or a non-transitory computer-readable storage media that store instructions that are executable by a computer processor that is connected to the sensor), and a second pressure sensor 122. In some embodiments, the first pressure sensor 121 may output a first measurement including an absolute ICP due to excess CSF, atmospheric pressure, and drift. In some embodiments, the second pressure sensor 122 may output a second measurement including an atmospheric pressure and drift. In some embodiments, the use of both the first pressure sensor 121 and the second pressure sensor 122 allows a corrected or accurate measurement of ICP (or drift corrected, absolute ICP). In some embodiments, the second pressure sensor 122 experiences the same drift as the first sensor 121, wherein any drift measured thereby can then be added or subtracted from the measurements of the first pressure sensor 121. In some embodiments, the first pressure sensor 121 is nearly drift free, making the second sensor 122 unnecessary. In some embodiments, the drift of the first pressure sensor and the drift of the second pressure sensor may be the same or similar.

The sensor drift from the first and/or second pressure sensors 121 and 122 may include any change in the output of the sensor(s) over time that may be inherent to any detection or sensing device. The drift may be caused by aging, temperature, humidity, environmental contamination, vibration, and others.

In some embodiments, the shunt system 100 may include an actuator that is coupled to and controls the valve 110. The actuator may be controlled by the controller which may automatically control the valve 110.

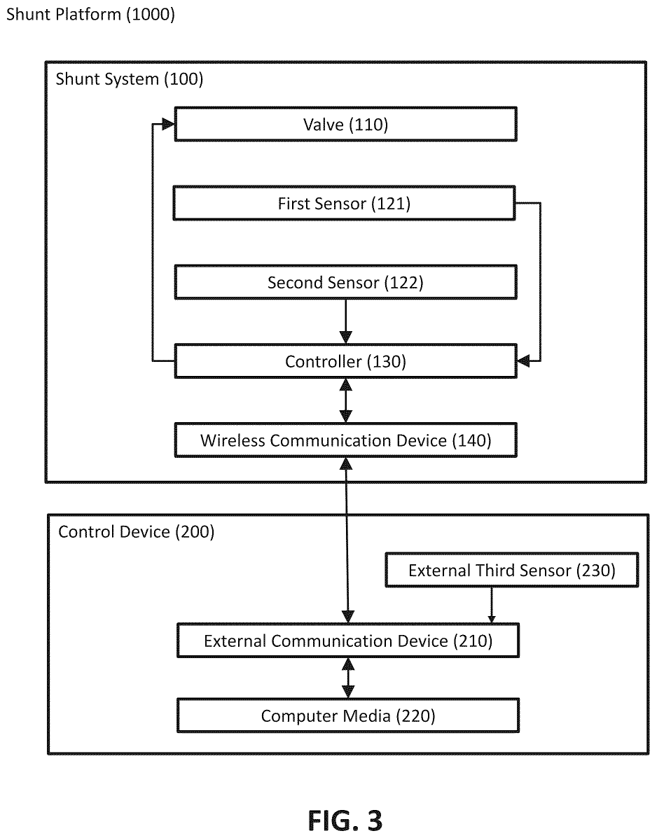

In some embodiments, the shunt system 100 further comprises a memory. In some embodiments, the memory may comprise the non-transitory computer readable media or be separate from the media. In some embodiments, per FIG. 3, the shunt system further comprises a wireless communication device 140. In some embodiments, the wireless communication device 140 comprises a Bluetooth device, a Wi-Fi device, Zigbee device, an NFC (Near Field Communication), a MedRadio device, a Z-Wave device, a Li-Fi device, an infrared device, a 2G device, a 3G device, a 4G device, or a 5G device. In some embodiments, the memory stores a plurality of ICP measurements including an absolute ICP measurement, an ICP measurement, a target ICP, atmospheric pressure, or any combination thereof.

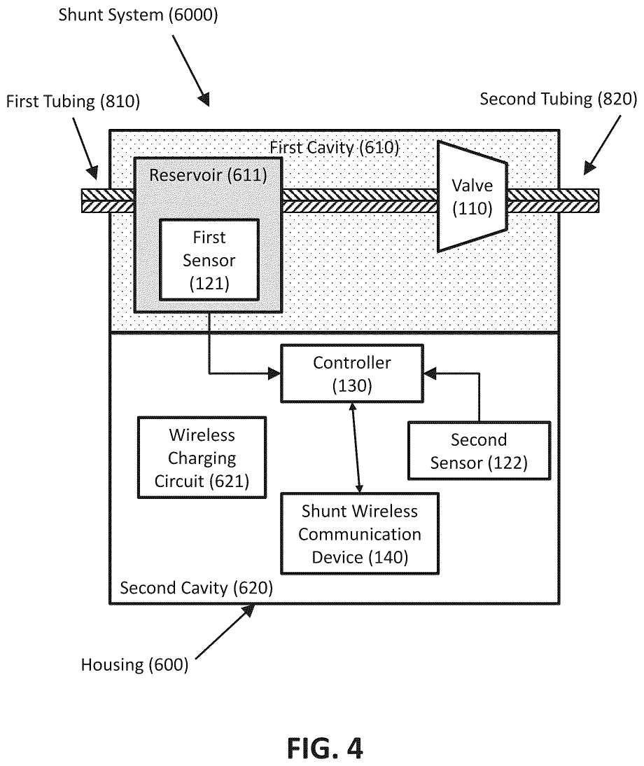

Another aspect provided herein includes a shunt system 6000 in FIG. 4 for monitoring ICP in a subject, in accordance with some embodiments. In some embodiments, the shunt system 6000 may comprise a first tubing 810 (proximal catheter), a valve 110, a second tubing 820 (distal catheter), a first pressure sensor 121 and a second pressure sensor 122. In some embodiments, the first tubing is configured to channel the CSF from the subject into the valve 110. In some embodiments, the valve 110 is coupled to the first tubing. In some embodiments, the valve 110 comprises a flow setting configured to control the flow of CSF. In some embodiments, the second tubing couples to the valve 110. In some embodiments, the second tubing is configured to excrete the CSF. In some embodiments, the first pressure sensor 121 is affixed to or integrated with the valve 110. In some embodiments, the first pressure sensor 121 is configured to sense an amount of absolute ICP. The amount of ICP may be related to the flow of the CSF. In some embodiments, the second sensor 212 is configured to sense an amount of drift by the second sensor 212 by measuring an atmospheric pressure within a second air-filled cavity. In some embodiments, the ICP may be a measurement of the CSF. The flow setting may be configured to change based on the sensed amount of ICP.

In some embodiments, the shunt system comprises a valve containing a volume of CSF and having a piezoelectric sensor coupled to a long catheter (e.g., first tubing) and a short catheter (e.g., second tubing), in accordance with some embodiments. In some embodiments, a microcontroller with a digital modulator may receive data from a drift collection sensor (the second sensor) comprising a piezoelectric sensor wherein the microcontroller adjusts a flow of the CSF. Further, in some embodiments, an inductive loop may provide wireless charging to a battery within the system and a wireless modulator transmits data to an external device (e.g., a wand device) having a memory, a digital circuit, a battery, and a display.

In some embodiments, the shunt system 100 herein may have a width or length of about 15 mm to about 70 mm, but are not limited thereto. The shunt system 100 herein may have a width or length of about 15 mm to about 20 mm, about 15 mm to about 25 mm, about 15 mm to about 30 mm, about 15 mm to about 35 mm, about 15 mm to about 40 mm, about 15 mm to about 45 mm, about 15 mm to about 50 mm, about 15 mm to about 55 mm, about 15 mm to about 60 mm, about 15 mm to about 65 mm, about 15 mm to about 70 mm, about 20 mm to about 25 mm, about 20 mm to about 30 mm, about 20 mm to about 35 mm, about 20 mm to about 40 mm, about 20 mm to about 45 mm, about 20 mm to about 50 mm, about 20 mm to about 55 mm, about 20 mm to about 60 mm, about 20 mm to about 65 mm, about 20 mm to about 70 mm, about 25 mm to about 30 mm, about 25 mm to about 35 mm, about 25 mm to about 40 mm, about 25 mm to about 45 mm, about 25 mm to about 50 mm, about 25 mm to about 55 mm, about 25 mm to about 60 mm, about 25 mm to about 65 mm, about 25 mm to about 70 mm, about 30 mm to about 35 mm, about 30 mm to about 40 mm, about 30 mm to about 45 mm, about 30 mm to about 50 mm, about 30 mm to about 55 mm, about 30 mm to about 60 mm, about 30 mm to about 65 mm, about 30 mm to about 70 mm, about 35 mm to about 40 mm, about 35 mm to about 45 mm, about 35 mm to about 50 mm, about 35 mm to about 55 mm, about 35 mm to about 60 mm, about 35 mm to about 65 mm, about 35 mm to about 70 mm, about 40 mm to about 45 mm, about 40 mm to about 50 mm, about 40 mm to about 55 mm, about 40 mm to about 60 mm, about 40 mm to about 65 mm, about 40 mm to about 70 mm, about 45 mm to about 50 mm, about 45 mm to about 55 mm, about 45 mm to about 60 mm, about 45 mm to about 65 mm, about 45 mm to about 70 mm, about 50 mm to about 55 mm, about 50 mm to about 60 mm, about 50 mm to about 65 mm, about 50 mm to about 70 mm, about 55 mm to about 60 mm, about 55 mm to about 65 mm, about 55 mm to about 70 mm, about 60 mm to about 65 mm, about 60 mm to about 70 mm, or about 65 mm to about 70 mm, including increments therein. The shunt system 100 herein may have a width or length of about 15 mm, about 20 mm, about 25 mm, about 30 mm, about 35 mm, about 40 mm, about 45 mm, about 50 mm, about 55 mm, about 60 mm, about 65 mm, or about 70 mm. The shunt system 100 herein may have a width or length of at least about 15 mm, about 20 mm, about 25 mm, about 30 mm, about 35 mm, about 40 mm, about 45 mm, about 50 mm, about 55 mm, about 60 mm, or about 65 mm. The shunt system 100 herein may have a width or length of at most about 20 mm, about 25 mm, about 30 mm, about 35 mm, about 40 mm, about 45 mm, about 50 mm, about 55 mm, about 60 mm, about 65 mm, or about 70 mm.

The shunt system 100 herein may have a height of about 0.2 mm to about 1.3 mm. The shunt system 100 herein may have a height of about 0.2 mm to about 0.3 mm, about 0.2 mm to about 0.4 mm, about 0.2 mm to about 0.5 mm, about 0.2 mm to about 0.6 mm, about 0.2 mm to about 0.7 mm, about 0.2 mm to about 0.8 mm, about 0.2 mm to about 0.9 mm, about 0.2 mm to about 1 mm, about 0.2 mm to about 1.1 mm, about 0.2 mm to about 1.2 mm, about 0.2 mm to about 1.3 mm, about 0.3 mm to about 0.4 mm, about 0.3 mm to about 0.5 mm, about 0.3 mm to about 0.6 mm, about 0.3 mm to about 0.7 mm, about 0.3 mm to about 0.8 mm, about 0.3 mm to about 0.9 mm, about 0.3 mm to about 1 mm, about 0.3 mm to about 1.1 mm, about 0.3 mm to about 1.2 mm, about 0.3 mm to about 1.3 mm, about 0.4 mm to about 0.5 mm, about 0.4 mm to about 0.6 mm, about 0.4 mm to about 0.7 mm, about 0.4 mm to about 0.8 mm, about 0.4 mm to about 0.9 mm, about 0.4 mm to about 1 mm, about 0.4 mm to about 1.1 mm, about 0.4 mm to about 1.2 mm, about 0.4 mm to about 1.3 mm, about 0.5 mm to about 0.6 mm, about 0.5 mm to about 0.7 mm, about 0.5 mm to about 0.8 mm, about 0.5 mm to about 0.9 mm, about 0.5 mm to about 1 mm, about 0.5 mm to about 1.1 mm, about 0.5 mm to about 1.2 mm, about 0.5 mm to about 1.3 mm, about 0.6 mm to about 0.7 mm, about 0.6 mm to about 0.8 mm, about 0.6 mm to about 0.9 mm, about 0.6 mm to about 1 mm, about 0.6 mm to about 1.1 mm, about 0.6 mm to about 1.2 mm, about 0.6 mm to about 1.3 mm, about 0.7 mm to about 0.8 mm, about 0.7 mm to about 0.9 mm, about 0.7 mm to about 1 mm, about 0.7 mm to about 1.1 mm, about 0.7 mm to about 1.2 mm, about 0.7 mm to about 1.3 mm, about 0.8 mm to about 0.9 mm, about 0.8 mm to about 1 mm, about 0.8 mm to about 1.1 mm, about 0.8 mm to about 1.2 mm, about 0.8 mm to about 1.3 mm, about 0.9 mm to about 1 mm, about 0.9 mm to about 1.1 mm, about 0.9 mm to about 1.2 mm, about 0.9 mm to about 1.3 mm, about 1 mm to about 1.1 mm, about 1 mm to about 1.2 mm, about 1 mm to about 1.3 mm, about 1.1 mm to about 1.2 mm, about 1.1 mm to about 1.3 mm, or about 1.2 mm to about 1.3 mm, including increments therein. The shunt system 100 herein may have a height of about 0.2 mm, about 0.3 mm, about 0.4 mm, about 0.5 mm, about 0.6 mm, about 0.7 mm, about 0.8 mm, about 0.9 mm, about 1 mm, about 1.1 mm, about 1.2 mm, or about 1.3 mm. The shunt system 100 herein may have a height of at least about 0.2 mm, about 0.3 mm, about 0.4 mm, about 0.5 mm, about 0.6 mm, about 0.7 mm, about 0.8 mm, about 0.9 mm, about 1 mm, about 1.1 mm, or about 1.2 mm. The shunt system 100 herein may have a height of at most about 0.3 mm, about 0.4 mm, about 0.5 mm, about 0.6 mm, about 0.7 mm, about 0.8 mm, about 0.9 mm, about 1 mm, about 1.1 mm, about 1.2 mm, or about 1.3 mm.

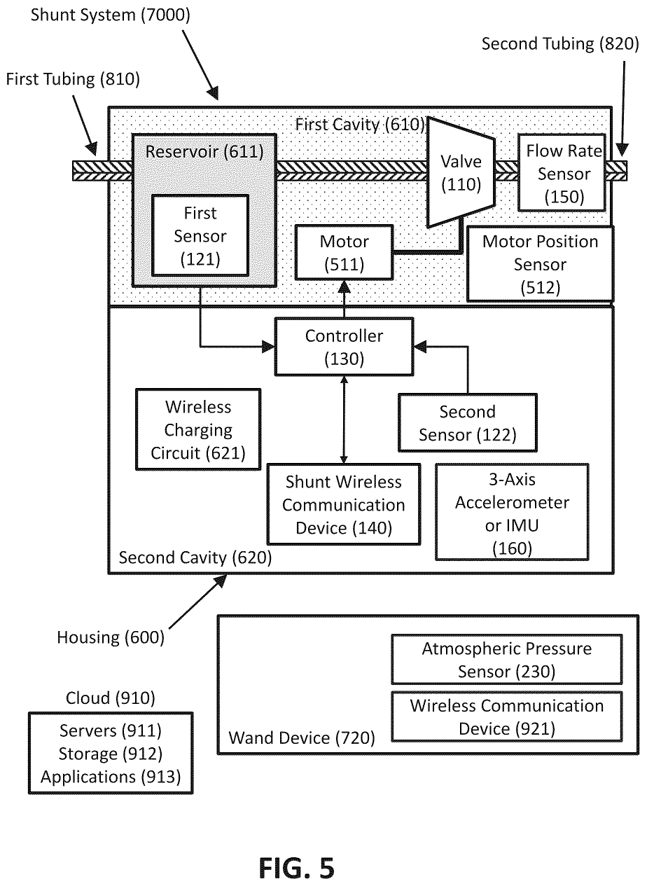

Further, provided herein per FIGS. 4-5, are shunt systems (6000, 7000) for monitoring ICP of CSF in a subject, in accordance with some embodiments. In some embodiments, per FIGS. 4 and 5, each of the systems 6000 and 7000 is similar to the shunt system 100 and comprises a valve 110, a ICP sensor 121, a second sensor 122, a controller 130, a flow rate sensor 150, and a shunt wireless communication device 140. In some embodiments, each of the valve 110, the ICP sensor 121, the flow rate sensor 150, the shunt wireless communication device 140, the controller 130, or any combination thereof is magnetic or non-magnetic.

In some embodiments, the valve 110 is configured to control a flow of the CSF in the subject. In some embodiments, per FIG. 5, the CSF flows through a ventricle tube 810 coupled to the ventricle of the subject, through the valve 110 of the shunt system (6000, 7000), and through a peritoneal tube 820 to the subject's peritoneum. In some embodiments, the valve 110 comprises one or more of a ball-spring valve 110, a floating ball valve 110, a trunnion mounted ball valve 110, a top entry ball valve 110, a side entry ball valve 110, a three-way ball valve 110, a ball check valve 110, a flapper check valve 110, a disc check valve 110, a ball relief valve 110, a spring safety valve 110, and a spring-loaded pressure relief valve 110. In some embodiments, the valve 110 is normally closed. In some embodiments, the valve 110 and the first pressure sensor 121 are integrated. In some embodiments, the valve 110 and the first sensor 121 are a single component.

In some embodiments, an opening pressure threshold of the valve 110 is adjustable. In some embodiments, the valve 110 is normally closed, wherein pressures of the CSF higher than the opening pressure threshold open the valve 110 to allow the CSF to flow therethrough. In some embodiments, the opening pressure threshold is adjustable between about 3 mmHg to about 40 mmHg. In some embodiments, the opening pressure threshold is adjustable between about 5 mmHg to about 25 mmHg.

In some embodiments, the ICP sensor 121 is configured to measure the ICP of the subject proximal to the valve 110. In some embodiments, the ICP sensor 121 is configured to measure a an absolute ICP of the subject. In some embodiments, the ICP sensor 121 is configured to measure the ICP of the subject within or proximal to the valve 110 and output the measured pressure, including the ICP, atmospheric pressure, and any drift of the ICP sensor 121. In some embodiments, the second sensor 122 is configured to measure an air pressure and may output the second measurement including the air pressure and any drift of the second sensor.

In some embodiments, ICP sensor 121 comprises: a capacitive sensor; a piezoelectric sensor; a piezoresistive sensor; a magnetic sensor; a resonant sensor; an optical sensor; a MEMS sensor. In some embodiments, the first sensor 121 and the second sensor 122 are the same type of sensor. In some embodiments, the first pressure sensor resides within the CSF fluid column and senses pressure exerted on the CSF that is flowing into or residing within the valve. In the event of shunt occlusion, there may not be CSF flow.

In some embodiments, the flow rate sensor 150 is configured to measure the flow rate of CSF through the valve 110. In some embodiments, the flow rate sensor comprises: a thermal flow rate sensor; a Lorentz Force Velocimetry sensor; or an electromagnetic sensor.

In some embodiments, the controller 130 is configured to calculate an absolute ICP based at least in part on a difference between the first and second measurements. In some embodiments, the controller 130 is further configured to cancel out a sensor drift of the first sensor 121. In some embodiments, the controller 130 is further configured to determine the sensor drift of the first sensor 121 based at least in part on the difference between the first measurement and the second measurement. In some embodiments, the first sensor 121 and the second sensor 122 being the same type of sensor enables improves drift calculation as both sensors are often prone to drift in the same direction over time by the same or similar amounts. In some embodiments, the drift is corrected by measuring an atmospheric pressure with a second pressure sensor proximal to the first pressure sensor, and a difference in pressure measurements is calculated to arrive at the drift corrected absolute ICP.

In some embodiments, the second sensor is positioned outside the fluid column in a cavity filled with atmospheric pressure air. In some embodiments, the drift and ICP are calculated with the following equations:

$$ICP = [ ( ICP + {P}_{atmosphere} + {Drift}_{1} ) - ( {P}_{0} + {Drift}_{2} ) - {P}_{atmosphere} ] + {P}_{0}$$

wherein the first measurement is (ICP+Pₐₜₘₒₛₚₕₑᵣₑ+Drift₁),
the second measurement is (P₀+Drift₂),
the third measurement is Pₐₜₘₒₛₚₕₑᵣₑ, (external to implanted system)
and the calibration at time of production removes the initial air pressure within the compartment of the implantable system, P₀, described herein, the last term in the ICP equation above

Assuming that the sensor drifts are equivalent (e.g., Drift₁ of first sensor and Drift₂ of second sensor are the same, or any difference between the two is negligible), Drift₁−Drift₂=0. ICP=first measurement-second measurement-third measurement.

In some embodiments, each sensor may be implemented to achieve nearly zero drift; thus, the drift may be assumed to be negligible. In this case, the equations above are modified as follows:

$$first measurement = ICP + {P}_{atmosphere}$$

$$second measurement = 0 ( after calibrating for {P}_{0} )$$

$$third measurement = {P}_{atmosphere} ( external to implanted device )$$

$$ICP = first measurement - third measurement$$

In some embodiments, the shunt wireless communication device 140 is configured to receive the ICP from the controller 130. In some embodiments, the shunt wireless communication device 140 is configured to transmit the ICP.

In some embodiments, per FIG. 5, the shunt system 7000 further comprises a motor 511 configured to adjust an opening pressure threshold of the valve 110. In some embodiments, the motor 511 comprises a DC motor, an AC motor, an induction motor, a synchronous motor, a stepper motor, or a servo motor.

In some embodiments, the controller 130 is further configured to direct the motor 511 to adjust the opening pressure threshold. In some embodiments, the controller 130 is further configured to receive a motor 511 position from the motor position sensor 512. In some embodiments, the controller 130 is further configured to receive a motor 511 position from the motor 511, wherein the motor 511 comprises a stepper motor 511. In some embodiments, the shunt wireless communication device 140 is further configured to receive a target opening pressure threshold, and wherein the controller 130 is further configured to control the motor 511 based at least in part on the target opening pressure threshold. In some embodiments, the controller 130 is further configured to receive a motor 511 position from the motor position sensor 511, wherein the shunt wireless communication device 140 is further configured to transmit an opening pressure threshold confirmation based on the motor 511 position.

In some embodiments, the shunt system (6000, 7000) and/or the sensor system 6001 further comprises a memory configured to store the ICP, the target ICP, or both. In some embodiments, the memory comprises a non-volatile memory.

In some embodiments, the shunt system (6000, 7000) and/or the sensor system 6001 further comprises a housing 600 comprising: a first cavity 610 comprising the first sensor 121 and the valve 110; and a second cavity 620 comprising the second sensor 122, wherein the second cavity 620 is isolated from the first cavity 610. In some embodiments, the second cavity 620 may be isolated from the first cavity 610 such that the second cavity 620 does not include any CSF. In some embodiments, the second cavity 620 may include the electronics described herein with respect to the second cavity and air. In some embodiments, the first cavity 610 further comprises a motor 511. In some embodiments, the second cavity 620 further comprises the controller 130 and the shunt wireless communication device 140. In some embodiments, as shown in FIG. 4, the first sensor 121 is located in a reservoir 611 in the first cavity 610 of the housing 600.

In some embodiments, a housing comprises a compartment and a conduit for the flow of CSF. A receptacle in the compartment may have an opening to the conduit such that the first pressure sensor may be secured in the receptacle so as to maintain isolation between the compartment and conduit. The first pressure sensor membrane and/or surface may come into contact with CSF in the conduit.

In some embodiments, the shunt system (6000, 7000) further comprises an energy storage device configured to power the valve 110, the ICP sensor 121, the controller 130, or any combination thereof. In some embodiments, the shunt system (6000, 7000) and/or the sensor system 6001 further comprises a wireless charging circuit 621 configured to receive power to charge the energy storage device, the controller 130, or both. In some embodiments, the energy storage device comprises a battery, a capacitor, a supercapacitor, or any combination thereof. In some embodiments, the energy storage device has a voltage of about 1 volt to about 10 volts. In some embodiments, the energy storage device has a voltage of at least about 1 volt, 2 volts, 3 volts, 4 volts, 5 volts, 6 volts, 7 volts, 8 volts, or 9 volts. In some embodiments, the energy storage device has a charge of at least about 500 mAh, 600 mAh, 700 mAh, 800 mAh, 900 mAh, 1,000 mAh, or more. In some embodiments, the energy storage device has a current of at least about 200 mA, 250 mA, 300 mA, 350 mA, 400 mA, 500 mA, or more. In some embodiments, the energy storage device has a voltage burst time of at least about 50 ms, 60 ms, 70 ms, 80 ms, 90 ms, 100 ms, 120 ms, 140 ms, or more.

In some embodiments, the shunt system (6000, 7000) and/or the sensor system 6001 further comprises a digital modulator configured to modulate a signal from the controller 130. In some embodiments, the controller 130 and the shunt wireless communication device 140 are communicably coupled by the digital modulator. In some embodiments, the shunt system (6000, 7000) and/or the sensor system 6001 further comprises a first analog to digital (A/D) converter communicably coupling the first sensor 121 and the controller 130 and configured to convert an analog signal from the first sensor 121 to a digital signal. In some embodiments, the shunt system (6000, 7000) and/or the sensor system 6001 further comprises a second A/D converter communicably coupling the second sensor 122 and the controller 130 and configured to convert an analog signal from the second sensor 122 to a digital signal. In some embodiments, the controller 130 comprises a digital modulator, one or more A/D converters, or both.

In some embodiments, the opening pressure threshold may be set based on a status of the subject of the shunt system. In some embodiments, measurements of the ICP may be taken while the subject is in the middle of an activity, and it may be beneficial to consider the subject's status before setting a target ICP so that the ICP is not based on a measurement taken during heightened physical activity (e.g., measurement taken while the subject was running). In some embodiments, the status comprises one or more of a sleeping status, an awake status, an exercising status, a standing status, a sitting status, a laying status, and a circadian status. As normal ICPs may vary based on the status of the subject, determination of the status enables improved ICP control to improve the health of the subject. In one example, as a subject's ICP may be higher while exercising, upon determination that the subject is in an exercising status, the controller 130 will increase the pressure threshold of the valve 110. In another example, as a subject's ICP may be lower while sleeping, upon determination that the subject is in a sleeping status, the controller 130 will decrease the pressure threshold of the valve 110. In some embodiments, the status of the subject may be determined by applying Fourier Analysis or signal processing techniques to the absolute ICP, the ICP, AIr, or any combination of one or more thereof. Applying Fourier Analysis can analyze the spectral content of the absolute ICP, the ICP, or both, to extract the harmonic content of the waveform. In some embodiments, the result of the analysis may provide the statuses when the subject has a high ICP and/or a low ICP while the subject is in the middle of different activities. In some embodiments, the time(s) may be used to determine when the target ICP should be set lower or higher based on the ICP values while the subject is engaging in that activity or has the status. Accelerometer 160 may be configured to measure the subject's position and the controller 130 may be configured to store the position at the time of ICP measurement, flow rate measurement, or any combination thereof.

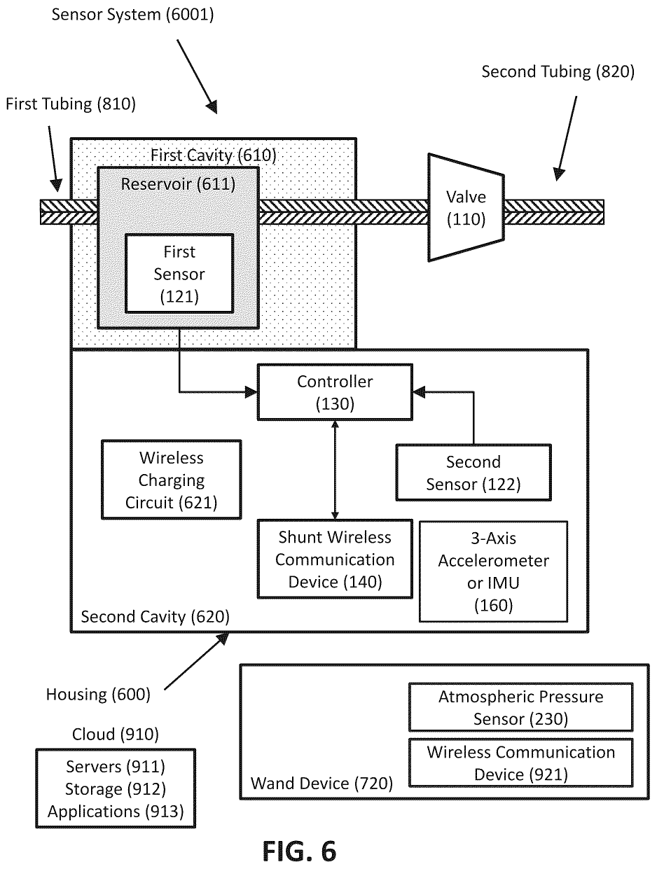

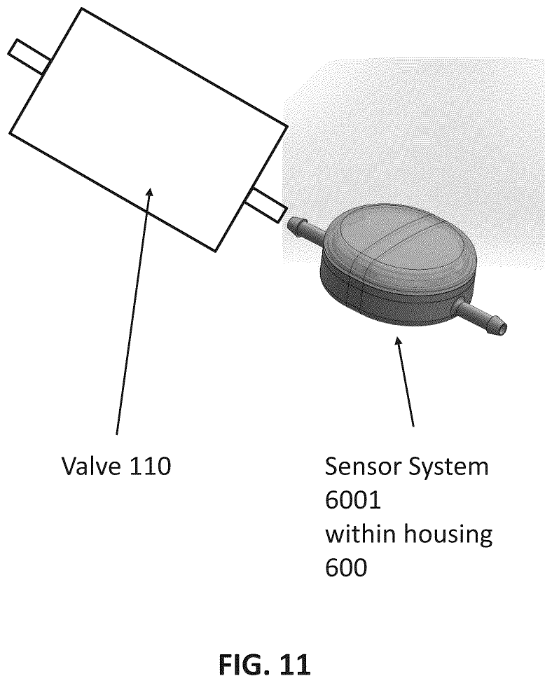

In some embodiments, per FIGS. 6, 11, and 14B, the shunt system may comprise a sensor system 6001, e.g., provided in a housing 600, that may not comprise the valve 110 disposed within the enclosed sensor system 6001. In some cases, the sensor system may be coupled to a valve 110 input external to the sensor system 6001. In some cases, the sensor system 6001 may be fluidically coupled to the valve 110.

### Operating Modes

In some embodiments, the shunt systems may operate in one or more operating modes. In some embodiments, the operating modes may include a manual mode, an adjust-assist mode, and/or an autonomous mode. In some embodiments, the shunt system 6000 may be configured to operate in the manual mode. In some embodiments, the shunt system 7000 may be configured to operate in the manual mode, adjust-assist mode, and/or the autonomous mode. In some embodiments, the shunt system 7000 may be configured to operate in any of the modes, depending on a user setting or configuration.

In some embodiments, in a manual mode of the shunt system 6000, per FIG. 4, an opening pressure threshold of the valve 110 is manually adjustable. In some embodiments, in the manual mode of the shunt system 6000, the opening pressure threshold of the valve 110 is manually adjustable by an external magnetic device. In some embodiments, in the manual mode of the shunt system 6000, the opening pressure threshold of the valve 110 is manually adjustable by placing the external magnetic device in proximity to the valve 110, in proximity to the controller 130, or both. In some embodiments, in the manual mode of the shunt system 6000, the opening pressure threshold of the valve 110 is manually adjustable by translating and/or rotating the external magnetic device in proximity to the valve 110, in proximity to the controller 130, or both.

In some embodiments, in an adjust-assist mode of the shunt system 7000, the controller 130 is further configured to adjust the opening pressure threshold of the valve 110. In some embodiments, in the adjust-assist mode of the shunt system 7000, the controller 130 is further configured to adjust the opening pressure threshold of the valve 110 based at least in part on the ICP, the absolute ICP, the atmospheric pressure, or any combination thereof. In some embodiments, in the adjust-assist mode, the controller 130 may receive a command or a signal from an external device to adjust a setting in the opening pressure threshold of the valve 110. For example, if the user of the external device desires a target ICP, the user may set the target ICP with the external device. Then, the external device may send a signal to the shunt system 7000 via the shunt wireless communication device 140 to set the opening pressure threshold to be a value that is consistent with the target ICP.

In some embodiments, per FIG. 5, in an autonomous mode of the shunt system 7000, the controller 130 is further configured to automatically adjust the opening pressure threshold of the valve 110 based on a feedback loop, in accordance with some embodiments. In some embodiments, the shunt system 7000 operating in autonomous mode may be configured to automatically adjust the opening pressure threshold of the valve whenever the controller detects that the ICP is below or more than a target ICP.

In some embodiments, there may be a table (e.g., in a memory of or connected to the controller) that includes a correspondence between a set of target ICPs and a set of target opening pressure thresholds. In some embodiments, if the valve's opening pressure threshold is set to one of the target opening pressure thresholds, the flow of CSF may be adjusted such that the ICP may be adjusted to be closer to or equal to the target ICP by adjusting the opening pressure threshold to be closer to or equal to the corresponding target opening pressure threshold. In some embodiments, when the controller detects that the ICP is less than the target ICP, the controller may automatically change the opening pressure threshold to be a target opening pressure threshold that corresponds to the target ICP. In some embodiments, when the controller detects the ICP to be greater than the target ICP, the controller may automatically change the opening pressure threshold to be a target opening pressure threshold that corresponds to the target ICP.

In some embodiments, in the autonomous mode of the shunt system 7000, the controller 130 may be further configured to adjust the opening pressure threshold of the valve 110 based on an algorithm that is based at least in part on artificial intelligence model, a machine learning model, a signal processing algorithm, etc.

In some embodiments, in the autonomous mode of the shunt system 7000, an algorithm is configured to determine the status of the subject based on the ICP, the first measurement, the second measurement, the third measurement, drift, accelerometer measurement, or any combination thereof. In some embodiments, in the autonomous mode of the shunt system 7000, the controller 130 is configured to automatically adjust the opening pressure threshold of the valve 110 based on the status. In some embodiments, an algorithm is configured to determine the status of the subject. In some embodiments, Fourier analysis or another signal processing technique, such as AIr, may be applied to one or more ICP waveforms which may include time series data output by the ICP sensor, flow rate sensor, etc. In some embodiments, an analysis may be performed to derive a spectral content of the first measurement, second measurement, third measurement, the ICP, or all of these, to extract a pattern of the data (e.g., spectral and harmonic content). In some embodiments, the output of the analysis may be used to train an algorithm. In some embodiments, a number of times the valve 110 opens per time unit (e.g., valve opening rate) may be measured or calculated and used to train the system. In some embodiments, the number of times the valve 110 opens is correlated to a volatility of the ICP and an associated subject status.

### Shunt Platforms

In one aspect disclosed herein, per FIG. 3, is a shunt platform 1000 for monitoring ICP of CSF in a subject, in accordance with some embodiments. In some embodiments, the platform 1000 comprises a shunt system 100 and a control device 200.

In some embodiments, the control device 200 is external to the subject's body and comprises a wireless communication device 210, a second non-transitory computer-readable storage media 220, and an atmospheric pressure sensor 230. In some embodiments, the control device 200 is wearable. In some embodiments, the control device 200 is worn by the subject. In some embodiments, the control device 200 is worn by a physician or medical technician. In some embodiments, the control device 200 is a smartphone. In some embodiments, the control device 200 further comprises a fastener. In some embodiments, the fastener comprises a strap, a band, a clamp, a bolt, a nut, a clasp, a hook and loop fastener, a cotter pin, a hitch pin, a clevis pin, a retaining ring, a spring clip, a magnet, a pin, or any combination thereof. In some embodiments, the control device 200 further comprises an atmospheric pressure sensor.

In some embodiments, the shunt system, the control device 200, or both further comprise an energy storage device powering the valve 110, the ICP sensor 121, the non-transitory computer-readable storage media, or any combination thereof. In some embodiments, the shunt system, the control device 200, or both further comprise a wireless charging circuit 621 (FIG. 5) receiving power to charge the energy storage device. In some embodiments, one or more of the components of the shunt system 100 is non-magnetic to allow the subject to undergo a magnetic resonance imaging (MRI) session during use.

Also provided herein, per FIGS. 5 and 6, is a platform for monitoring ICP of CSF in a subject, in accordance with some embodiments. In some embodiments, the platform comprises the shunt system (6000, 7000) and/or sensor system 6001 herein and an external wand device 720.

In some embodiments, the wand device 720 comprises a wand wireless communication device 921. In some embodiments, the wand wireless communication device 921 is configured to receive the ICP from the shunt wireless communication device 140. In some embodiments, the wand wireless communication device 921 is configured to receive the ICP from the shunt wireless communication device 140 when the wand device 720 is within a set proximity to the shunt system (6000, 7000) and/or the sensor system 6001. In some embodiments, the wand wireless communication device 921 is further configured to transmit a target opening pressure threshold to the shunt wireless communication device 140. In some embodiments, the wand wireless communication device 921 is further configured to transmit the atmospheric pressure to the shunt wireless communication device 140. In some embodiments, the wand wireless communication device 921 is further configured to transmit a target opening pressure threshold to the shunt wireless communication device 140 when the wand device 720 is within a set proximity to the shunt system (6000, 7000) and/or the sensor system 6001.

In some embodiments, the shunt system (6000, 7000) and/or the sensor system 6001 further comprises a wireless charging circuit 621 configured to receive power from the wand device 720 to charge the energy storage device, the controller 130, or both, when the wand device 720 is within a set proximity to the shunt system (6000, 7000) and/or the sensor system 6001.

In some embodiments, once the wand device 720 is outside the set proximity to the shunt system (6000, 7000) and/or the sensor system 6001, the wireless charging circuit 621 stops receiving power from the wand device 720. In some embodiments, once the wand device 720 is outside the set proximity to the shunt system (6000, 7000), the shunt's motor 511 remains in position. In some embodiments, the motor 511 comprises a stepper motor 511 with a detent torque, such that, once the wand device 720 is outside the set proximity to the shunt system (6000, 7000), the shunt's motor 511 remains in position.

In some embodiments, once the wand device 720 is outside the set proximity to the shunt system (6000, 7000) and/or the sensor system 6001, a current state of the shunt system (6000, 7000) and/or the sensor system 6001 is saved to its memory. In some embodiments, the current state comprises motor 511-determined position, a motor 511 position determined by the controller 130, the ICP, or any combination thereof. In some embodiments, the set proximity is about 1 inch, 1.5 inches, 2 inches, 2.5 inches, 3 inches, 3.5 inches, 4 inches, 4.5 inches, 5 inches, or more. In some embodiments, the reduced set proximity enables the use of a wireless charging circuit 621 with lower gain, size, and power consumption.

In some embodiments, the wand wireless communication device 921 is further configured to receive the opening pressure threshold confirmation. In some embodiments, the wand wireless communication device 921 is further configured to transmit the target opening pressure. In some embodiments, the shunt wireless communication device 140 is further configured to receive the target opening pressure threshold. In some embodiments, the controller 130 is configured to direct the motor 511 based on the target opening pressure threshold.

In some embodiments, the shunt system (6000, 7000) further comprises an energy storage device configured to power the valve 110, the ICP sensor 121, the controller 130, or any combination thereof. In some embodiments, the shunt system (6000, 7000) and/or the sensor system 6001 further comprises a wireless charging circuit 621 configured to receive power to charge the energy storage device, the controller 130, or both. In some embodiments, the energy storage device comprises a battery, a capacitor, a supercapacitor, or any combination thereof. In some embodiments, the shunt system (6000, 7000) and/or the sensor system 6001 further comprises a wireless charging circuit 621 configured to receive power from the wand device 720 to charge the energy storage device, the controller 130, or both.

In some embodiments, the wand device 720 further comprises a display. In some embodiments, the display is configured to show data based on the absolute ICP, the ICP, the atmospheric pressure, the opening pressure threshold confirmation, the target opening pressure, or any combination thereof.

In some embodiments, the wand device 720 further comprises a speaker. In some embodiments, the speaker is configured to emit a sound based on the absolute ICP, the ICP, the atmospheric pressure, the opening pressure threshold confirmation, the target opening pressure, or any combination thereof.

In some embodiments, per FIG. 5, the wand wireless communication device 921 is further configured to transmit data including the absolute ICP, the ICP, the opening pressure threshold confirmation, rate of valve opening, pattern characteristics of valve opening, or any combination thereof to a cloud storage 910. In some embodiments, as shown, the cloud storage 910 comprises servers 911, storage 912, and applications 913.

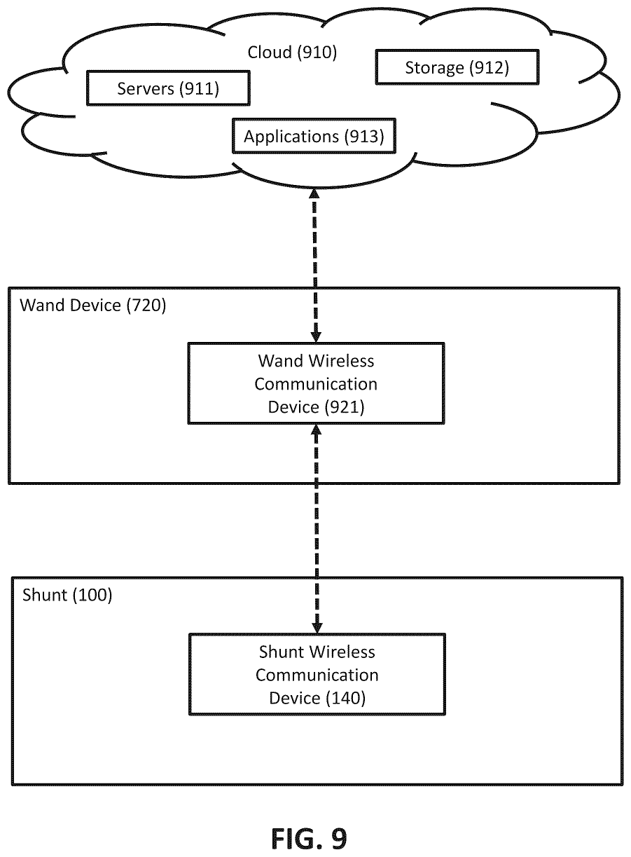

FIG. 9 shows a diagram of an exemplary shunt platform with cloud connectivity. A cloud 910 comprising servers 911, storage 912, and applications 913 may be connected to a wand wireless communication device 921, which may be part of the wand device 720. The wand wireless communication device 921 may be connected to a shunt wireless communication device 140, which may be part of the shunt 100. The shunt wireless communication device 140 and the wand wireless communication device 921 may all connect to and communicate with the cloud 910.

In some embodiments, the ICP sensor is integrated with the valve. In some embodiments, a channel of the valve comprises a cavity that accepts the ICP sensor, such that the ICP sensor measures the ICP of the subject at a point where the CSF flows through the valve. In some embodiments, the flow setting has a plurality of setpoints that change the flow of the CSF, and wherein the flow setting is configured to step-up or step-down only one setpoint over the predetermined period of time. For example, increasing and decreasing the setpoints of the flow may be controlled so that there is not too much change in the flow. In some embodiments, the predetermined period of time may be preprogrammed or may be adjusted so that the change is controlled. In some embodiments, the valve is programmable to have one of a plurality of setpoints.

In some embodiments, the system further comprises a memory configured to store the plurality of flow settings and/or the sensed amount of ICP. In some embodiments, the memory is disposed outside the subject. In some embodiments, the memory includes a random-access memory (RAM) or an electrically erasable programmable read-only memory (EEPROM), and wherein the flow setting of the valve is configured to change based on data read from the memory. In some embodiments, the memory is configured to store pressure values over time. In some embodiments, the system further comprises a control device including a 3-axis gyroscope, accelerometer, or inertial measurement unit (IMU), the control device worn by the subject, wherein the control device is configured to detect the subject's position based at least in part on the accelerometer. In some embodiments, an amount of change in the ICP is less than a predetermined ICP threshold over a predetermined period of time.

In some embodiments, the system further comprises a controller configured to detect a change in the amount of ICP and change the flow setting of the valve based on the change in the amount of ICP. In some embodiments, the controller is further configured to wirelessly connect to a user device and transmit information including the flow setting of the programmable valve. In some embodiments, the controller includes a microcontroller or a microprocessor. In some embodiments, the controller includes a processor. In some embodiments, the controller is configured to communicate with an external device (e.g., a wand device or a smartphone) which may be worn by the subject. In some embodiments, the external device is disposed on a table or attached to a belt worn by the subject.

In some embodiments, the system further comprises a transmitter electrically connected to the sensor and configured to transmit any relevant ICP data to an external device. In some embodiments, the output of the sensor is passed through an analog-to-digital converter. In some embodiments, the sensor is configured to provide an output, and the transmitter is configured to convert and modulate the output and transmit the output via wireless signal. In some embodiments, the output provided by the sensor is a voltage or a current that indicates the pressure value. In some embodiments, the external device is configured to demodulate the received output from the transmitter to recover the pressure value. In some embodiments, the transmitter is configured to modulate the signal via ASK, FSK, QPSK, AM or FM. In some embodiments, the transmitter is configured to modulate the signal using the Bluetooth, low energy Bluetooth (BLE) or Near Field Communication protocols. In some embodiments, the external device comprises a smartphone, a wearable wireless device, a tablet, or computer. In some embodiments, the sensor includes a microelectromechanical system (MEMS) device or a piezo-electric device.

In some embodiments, the system further comprises a motor connected to the valve and configured to adjust the flow setting of the valve and thus the flow of CSF. In some embodiments, the system further comprises a rechargeable charge storage element configured to be charged wirelessly. In some embodiments, the system further comprises an external device configured to wirelessly charge the rechargeable charge storage element. In some embodiments, the rechargeable charge storage element includes a rechargeable battery or a capacitor. In some embodiments, the system further comprises an external device configured to power the sensor during reading. In some embodiments, the valve is composed of non-ferromagnetic material. In some embodiments, the flow setting of the valve is determined based on a machine learning model. In some embodiments, the machine learning module determines a status of the subject, wherein the controller adjusts the flow of the valve based on the status of the subject. In some embodiments, the status comprises an awake status, a sleeping status, an active status, an exercising status, a sitting status, a standing status, a laying status, or any combination thereof.

### Valves

In some embodiments, the valve controls a flow of CSF in the subject. In some embodiments, the valve and the ICP sensor are integrated. In some embodiments, the ICP sensor is communicatively affixed to the valve such that the ICP sensor can detect the pressure at or near the valve. In some embodiments, the valve comprises a Ball valve, a butterfly valve, a gate valve, a globe valve, a check valve, a diaphragm valve, a needle valve, a pressure relief valve, a plug valve, a solenoid valve, an angle valve, a control valve, a foot valve, a knife valve, a pinch valve, a rotary valve, a swing valve, a three-way valve, a wafer valve, a Y-Strainer valve, or any combination thereof. The valve may have a plurality of flowrate set points, wherein each set point corresponds to a different flow.

### Sensors

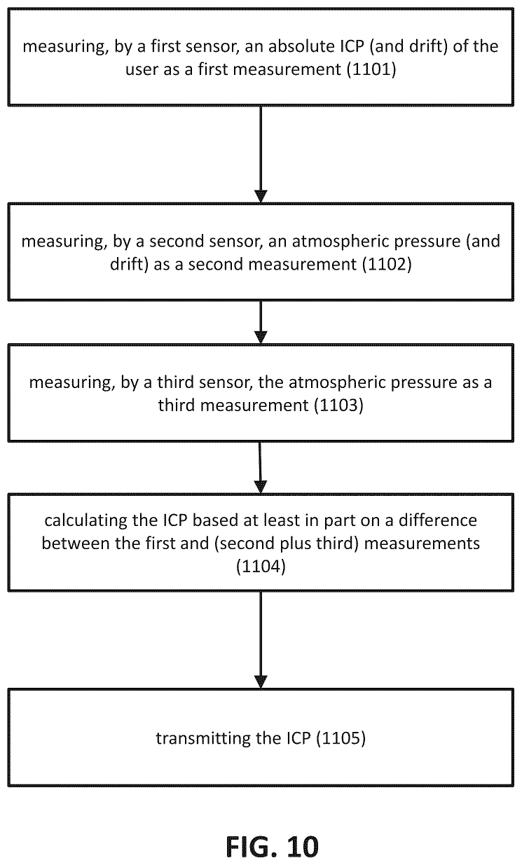

Another aspect provided herein, per FIG. 10, is a computer-implemented method for the ICP of the CSF in a subject. In some embodiments, the method comprises measuring, by an ICP sensor, an absolute ICP of the subject 1101; measuring, by a second sensor 1102, an atmospheric pressure with its own drift that is substantially the same as the drift of the first sensor 1101, calculating a drift corrected absolute ICP based at least in part on a difference between the first and second sensor measurements; and measuring, by a third sensor 1103, the atmospheric pressure and relaying this to the shunt wireless device 140. The ICP may be calculated at least in part on a difference between the drift corrected absolute ICP and the atmospheric pressure 1103 to produce the ICP 1104 which is then transmitted 1105.

In some embodiments, the method further comprises determining a sensor drift of the ICP sensor based at least in part on the difference between the measured absolute ICP and the measured drift of the calibrated second sensor. In some embodiments, the method further comprises storing the target opening pressure threshold to a memory. In some embodiments, the method further comprises storing the absolute ICP, the ICP, the atmospheric pressure, or any combination thereof in a memory.

In some embodiments, the method further comprises adjusting an opening pressure of a valve. In some embodiments, in a manual mode of the shunt system, the adjustment is performed manually. In some embodiments, in the manual mode of the shunt system, the adjustment is performed by a magnetic device. In some embodiments, the adjustment is performed by a motor. In some embodiments, the method further comprises receiving a target opening pressure threshold. In some embodiments, in an adjust-assist mode of the shunt system, the method further comprises receiving a target opening pressure threshold, wherein the adjustment is performed based on the target opening pressure threshold.

In some embodiments, in an autonomous mode of the shunt system, the adjustment is performed based at least in part on the ICP, the absolute ICP, the measured atmospheric pressure, or any combination thereof. In some embodiments, the adjustment is performed based on a machine learning model. In some embodiments, the machine learning model is configured to determine a status of the subject based on the ICP, the absolute ICP, the measured atmospheric pressure, or any combination thereof. In some embodiments, the status comprises one or more of a sleeping status, an awake status, an exercising status, a standing status, a sitting status, a laying status, and a circadian status. In some embodiments, the machine learning model is configured to determine the status of the subject by applying Fourier Analysis or signal processing techniques. In some embodiments, the machine learning model is configured to determine the status of the subject by counting a number of times the valve opens per unit time (opening rate).

In some embodiments, in the autonomous mode of the shunt system, the opening pressure threshold is increased by at most one increment. In some embodiments, the increment is a step width of the stepper motor. In some embodiments, the autonomous mode of the shunt system is only implemented once a sufficient number of ICP measurements are recorded to train the machine learning model. In some embodiments, in the autonomous mode, the method further comprises reverting the opening pressure threshold to a base opening pressure threshold if a charge of the energy storage device of the shunt system falls below a charge threshold. In some embodiments, the base opening pressure threshold is predetermined. In some embodiments, the base opening pressure threshold is set by a clinician. In some embodiments, reverting the opening pressure threshold to a base opening pressure threshold prevents the system from remaining at an opening pressure threshold set based off a past user status once their status has changed. For example, if in the autonomous mode, the opening pressure threshold may be set for a sleeping status and the battery is depleted before an exercise status is detected, reverting the opening pressure threshold to the base opening pressure threshold prevents harm to the subject while they exercise or engage in other activity.

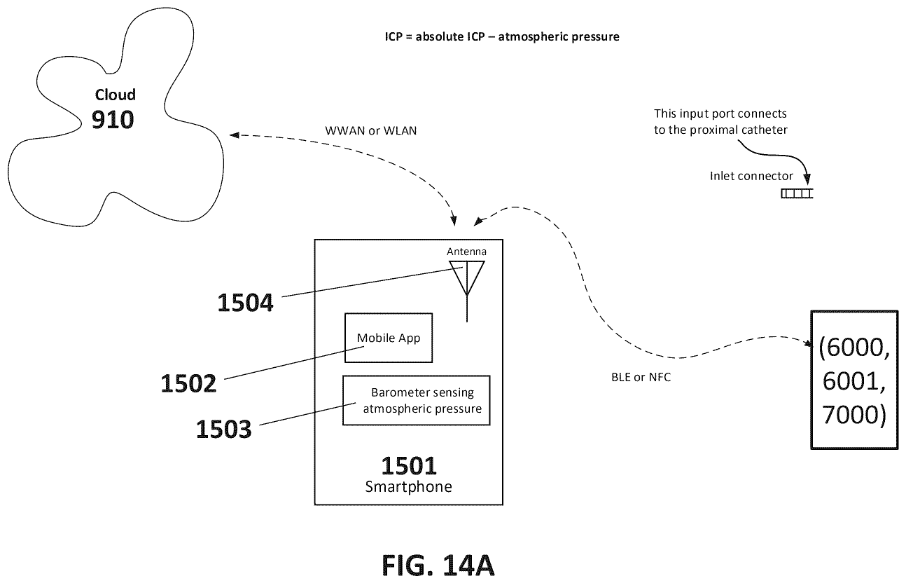

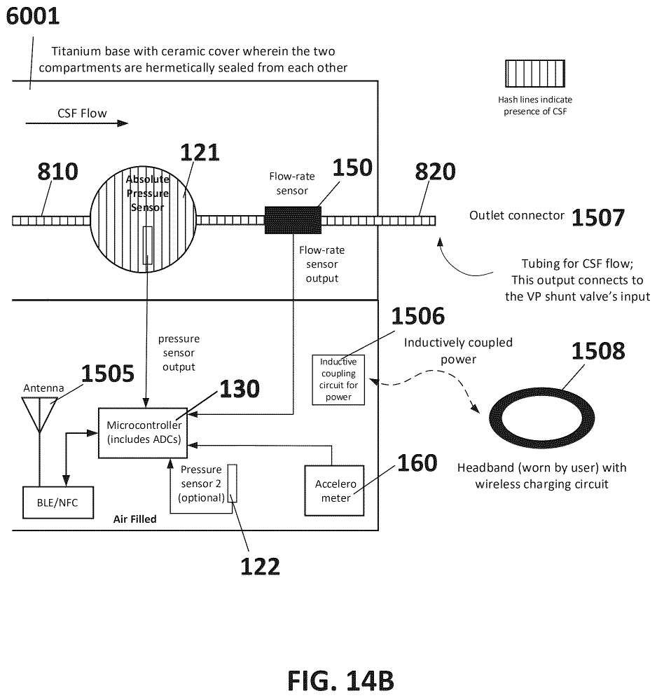

FIGS. 14A-14B illustrate a diagram of the implantable system described herein. As shown in FIG. 14A, the cloud 910 may be connected to a smartphone 1501. The smartphone 1501 may comprise a mobile app 1502, a barometer sensing atmospheric pressure 1503, and/or a smartphone antenna 1504. The cloud may be connected to the smartphone via a WWAN or WLAN connection.

FIG. 14B is a continuation of FIG. 14A from the right-hand side of FIG. 14A to the left-hand side of FIG. 14B. As shown in FIGS. 14A-14B, the smartphone may be connected to the shunt system (6000, 7000) and/or a sensor system 6001 via low-energy Bluetooth (BLE) or NFC. The inlet connector 1505 may connect to the proximal catheter 810. The shunt system (6000, 7000) and/or the sensor system 6001 may comprise a titanium base with a ceramic cover, wherein two compartments may be hermetically sealed from each other. In the first compartment, the proximal catheter 810 may connect to the absolute pressure sensor 121, which may connect to the flow rate sensor 150. CSF may flow through the first compartment. The flow rate sensor may connect to tubing for CSF flow 820. The tubing for CSF flow 820 may connect to an outlet connector 1507. The tubing for CSF flow 820 may connect to the VP shunt valve's input. The absolute pressure sensor 121 output and the flow rate sensor 150 output may be routed to the microcontroller 130, which may be located in the second compartment. The smartphone antenna 1504 may communicate the shunt system antenna 1505, which may use BLE/NFC to communicate with the microcontroller 130. The accelerometer 160 may also communicate with the microcontroller 130. The second compartment containing the microcontroller 130 and the accelerometer 160 may also be filled with air. The second compartment also may optionally comprise a second pressure sensor 122, which may also communicate with the microcontroller. The microcontroller may also include ADCs. The second compartment may also include an inductive coupling circuit for power 1506. The inductive

coupling circuit for power 1506 may communicate by inductive power to a headband 1508 worn by the subject, which may have a wireless charging circuit. The hash lines in the diagram indicate the presence of CSF, which may flow through (left to right) the proximal catheter 810, the absolute pressure sensor 121, the flow rate sensor 150, and finally through the tubing for CSF flow 820 to the outlet connector 1507.

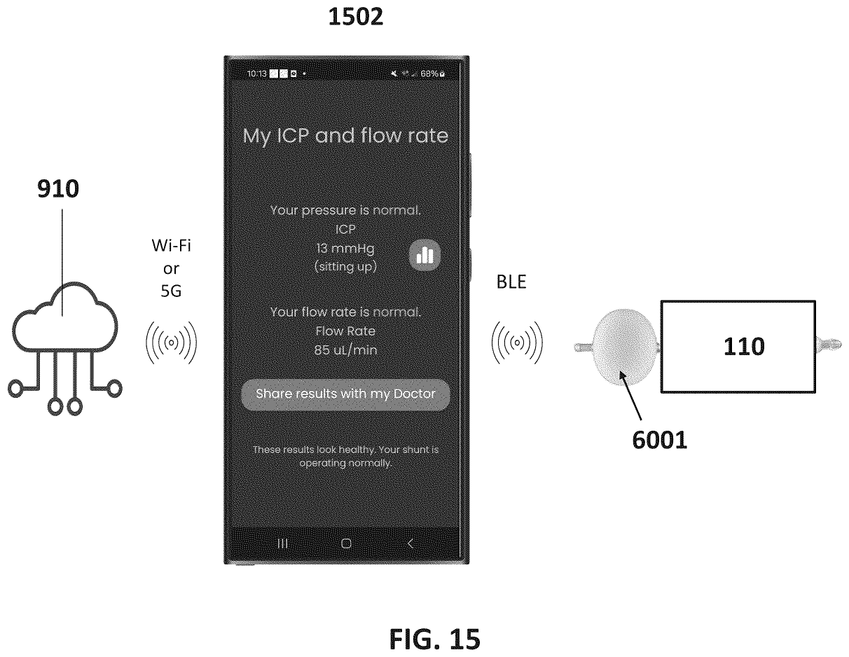

FIG. 15 illustrates the wireless connections between the cloud 910, the smartphone app 1502, and the valve 110. The cloud 910 may communicate with the smartphone app 1502 by a WiFi or 5G connection. The smartphone app 1502 may communicate with the valve 110 by BLE.

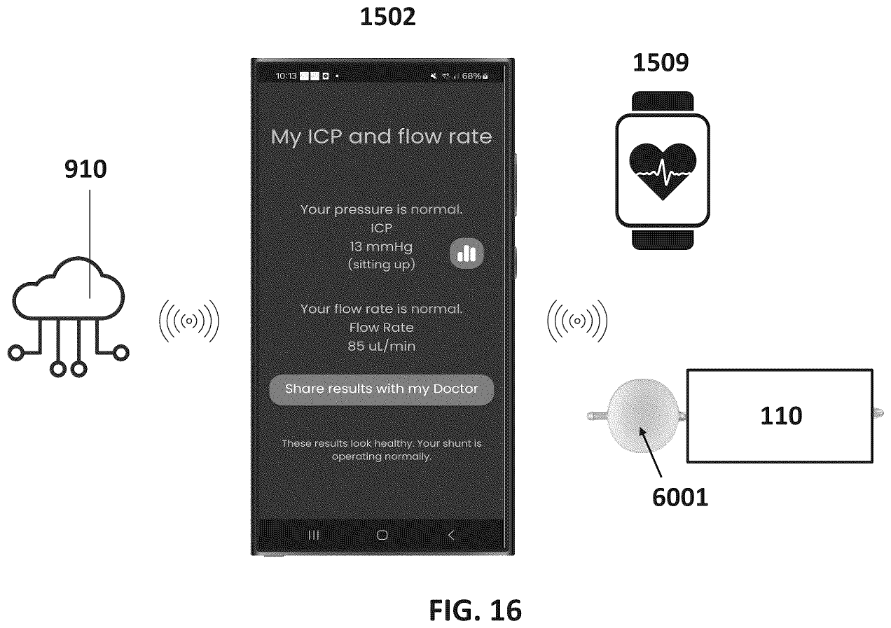

FIG. 16 shows a diagram of how a heart rate as measured by a smartwatch or other wearable device may be integrated with ICP and flow rate as measured by the implantable system described herein. The cloud 910 may communicate with the smartphone app 1502. The smartphone app 1502 may communicate with the wearable device 1509.

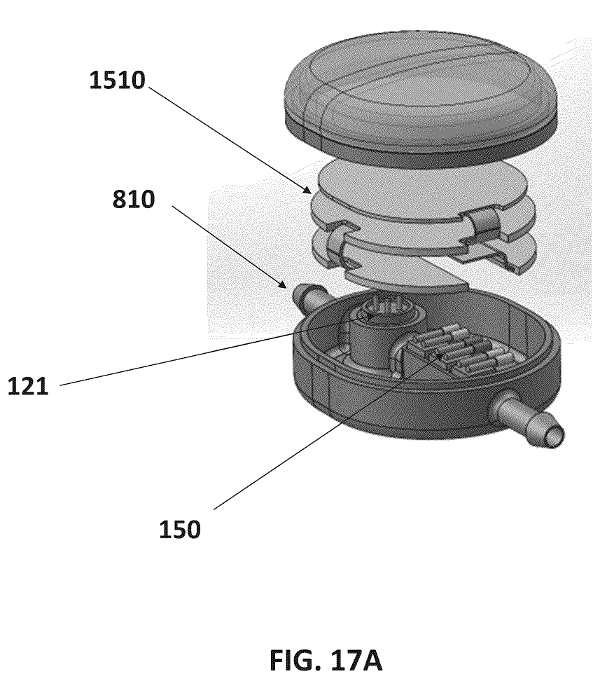

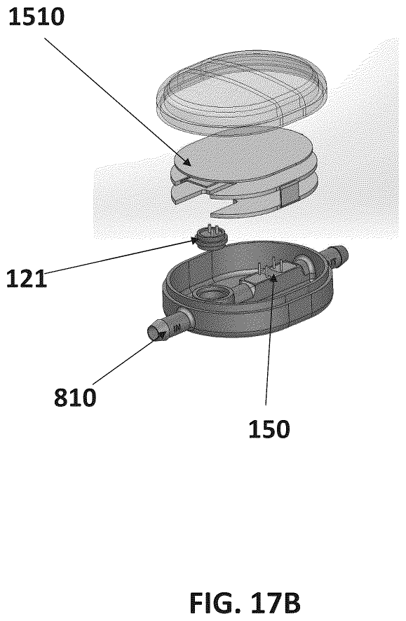

Table 2 summarizes example features of the implantable system described herein. FIGS. 17A-17B show a perspective of some different components within the implantable system described herein, including the primary pressure sensor 121, a tube that CSF flows through 810, a flow rate sensor 150, and a printed circuit board 1510 on which electronics are mounted. The only opening in the tube that the CSF flows through 810 may be where the pressure sensor is mounted to allow the sensor membrane and/or surface to contact the fluid. As mentioned elsewhere herein, the implantable system may comprise a titanium base with a ceramic cover. However, the implantable system may comprise other materials.

TABLE 2
Summary of example features of implantable system.

| System Feature | Details | Benefit |
|---|---|---|
| Noninvasive ICP | Continuous measurement | Check on shunt health at home |
| Noninvasive Flow Rate | Continuous measurement | Assess shunt patency; important for |
| | | NPH assessment |
| VP Shunt Health Status | Status modes: normal, high | Speeds diagnosis; minimizes x-ray |
| | ICP, low ICP, occlusion | exposure |
| Wireless Link to Smartphone | Mobile app | Enables data analytics and AI in the |
| | | cloud; works at scale with common |
| | | devices |
| Wireless Charging | Inductive coupling | No battery implanted |
| Pressure Reading | Accelerometer gates data | Ensures integrity of data |
| Stabilization | during motion. | |
| Body Temperature | High precision reading | Check for infection |
| Heart Rate | Synchronous with ICP and | Check for infection |
| | flow data | |

### Computing Systems

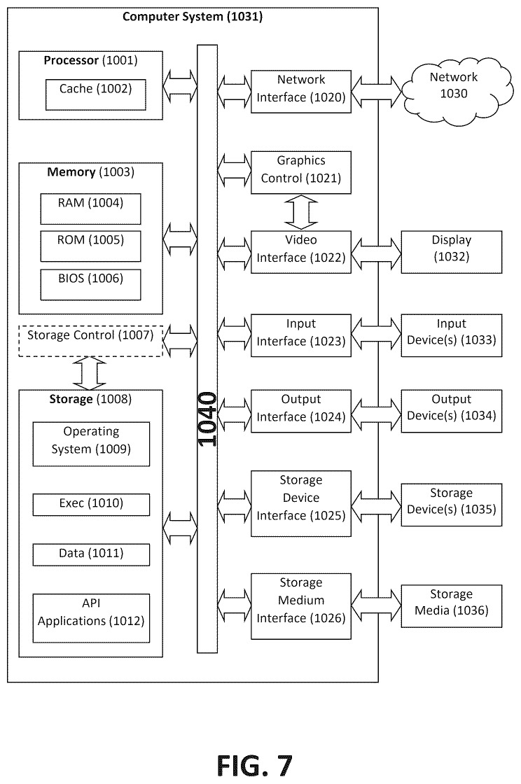

Referring to FIG. 7, a block diagram is shown depicting an exemplary machine that includes a computer system 1031 (e.g., a processing or computing system) within which a set of instructions can execute for causing a device to perform or execute any one or more of the aspects and/or methodologies for static code scheduling of the present disclosure. The components in FIG. 7 are examples only and do not limit the scope of use or functionality of any hardware, software, embedded logic component, or a combination of two or more such components implementing particular embodiments.

Computer system 1031 may include one or more processors 1001, a memory 1003, and a storage 1008 that communicate with each other, and with other components, via a bus 1040. The bus 1040 may also link a display 1032, one or more input devices 1033 (which may, for example, include a keypad, a keyboard, a mouse, a stylus, etc.), one or more output devices 1034, one or more storage devices 1035, and various tangible storage media 1036. All of these elements may interface directly or via one or more interfaces or adaptors to the bus 1040. For instance, the various tangible storage media 1036 can interface with the bus 1040 via storage medium interface 1026. Computer system 1031 may have any suitable physical form, including but not limited to one or more integrated circuits (ICs), printed circuit boards (PCBs), mobile handheld devices (such as mobile telephones or PDAs), laptop or notebook computers, distributed computer systems, computing grids, or servers.

Computer system 1031 includes one or more processor(s) 1001 (e.g., central processing units (CPUs) or general-purpose graphics processing units (GPUs)) that carry out functions. Processor(s) 1001 optionally comprise a cache memory unit 1002 for temporary local storage of instructions, data, or computer addresses. Processor(s) 1001 are configured to assist in execution of computer readable instructions. Computer system 1031 may provide functionality for the components depicted in FIG. 7 as a result of the processor(s) 1001 executing non-transitory, processor-executable instructions embodied in one or more tangible computer-readable storage media, such as memory 1003, storage 1008, storage devices 1035, and/or storage medium 1036. The computer-readable media may store software that implements particular embodiments, and processor(s) 1001 may execute the software. Memory 1003 may read the software from one or more other computer-readable media (such as mass storage device(s) 1035, 1036) or from one or more other sources through a suitable interface, such as network interface 1020. The software may cause processor(s) 1001 to carry out one or more processes or one or more steps of one or more processes described or illustrated herein. Carrying out such processes or steps may include defining data structures stored in memory 1003 and modifying the data structures as directed by the software.

The memory 1003 may include various components (e.g., machine readable media) including, but not limited to, a random-access memory component (e.g., RAM 1004) (e.g., static RAM (SRAM), dynamic RAM (DRAM), ferroelectric random-access memory (FRAM), phase-change random access memory (PRAM), etc.), a read-only memory component (e.g., ROM 905), and any combinations thereof. ROM 1005 may act to communicate data and instructions unidirectionally to processor(s) 1001, and RAM 1004 may act to communicate data and instructions bidirectionally with processor(s) 1001. ROM 1005 and RAM 1004 may include any suitable tangible computer-readable media described below. In one example, a basic input/output system 1006 (BIOS), including basic routines that help to transfer information between elements within computer system 1031, such as during start-up, may be stored in the memory 1003.

Fixed storage 1008 is connected bidirectionally to processor(s) 1001, optionally through storage control unit 1007. Fixed storage 1008 provides additional data storage capacity and may also include any suitable tangible computer-readable media described herein. Storage 1008 may be used to store operating system 1009, executable(s) 1010, data 1011, applications 1012 (application programs), and the like. Storage 1008 can also include an optical disk drive, a solid-state memory device (e.g., flash-based systems), or a combination of any of the above. Information in storage 1008 may, in appropriate cases, be incorporated as virtual memory in memory 1003.

In one example, storage device(s) 1035 may be removably interfaced with computer system 1031 (e.g., via an external port connector (not shown)) via a storage device interface 1025. Particularly, storage device(s) 1035 and an associated machine-readable medium may provide non-volatile and/or volatile storage of machine-readable instructions, data structures, program modules, and/or other data for the computer system 1031. In one example, software may reside, completely or partially, within a machine-readable medium on storage device(s) 1035. In another example, software may reside, completely or partially, within processor(s) 1001.

Bus 1040 connects a wide variety of subsystems. Herein, reference to a bus may encompass one or more digital signal lines serving a common function, where appropriate. Bus 1040 may be any of several types of bus structures including, but not limited to, a memory bus, a memory controller, a peripheral bus, a local bus, and any combinations thereof, using any of a variety of bus architectures. As an example and not by way of limitation, such architectures include an Industry Standard Architecture (ISA) bus, an Enhanced ISA (EISA) bus, a Micro Channel Architecture (MCA) bus, a Video Electronics Standards Association local bus (VLB), a Peripheral Component Interconnect (PCI) bus, a PCI-Express (PCI-X) bus, an Accelerated Graphics Port (AGP) bus, HyperTransport (HTX) bus, serial advanced technology attachment (SATA) bus, and any combinations thereof.

Computer system 1031 may also include an input device 1033. In one example, a user of computer system 1031 may enter commands and/or other information into computer system 1031 via input device(s) 1033. Examples of an input device(s) 1033 include, but are not limited to, an alpha-numeric input device (e.g., a keyboard), a pointing device (e.g., a mouse or touchpad), a touchpad, a touch screen, a multi-touch screen, a joystick, a stylus, a gamepad, an audio input device (e.g., a microphone, a voice response system, etc.), an optical scanner, a video or still image capture device (e.g., a camera), and any combinations thereof. In some embodiments, the input device is a Kinect, Leap Motion, or the like. Input device(s) 1033 may be interfaced to bus 1040 via any of a variety of input interfaces 1023 (e.g., input interface 1023) including, but not limited to, serial, parallel, game port, USB, FIREWIRE, THUNDERBOLT, or any combination of the above.

In particular embodiments, when computer system 1031 is connected to network 1030, computer system 1031 may communicate with other devices, specifically mobile devices and enterprise systems, distributed computing systems, cloud storage systems, cloud computing systems, and the like, connected to network 1030. Communications to and from computer system 1031 may be sent through network interface 1020. For example, network interface 1020 may receive incoming communications (such as requests or responses from other devices) in the form of one or more packets (such as Internet Protocol (IP) packets) from network 1030, and computer system 1031 may store the incoming communications in memory 1003 for processing. Computer system 1031 may similarly store outgoing communications (such as requests or responses to other devices) in the form of one or more packets in memory 1003 and communicated to network 1030 from network interface 1020. Processor(s) 1001 may access these communication packets stored in memory 1003 for processing.

Examples of the network interface 1020 include, but are not limited to, a network interface card, a modem, and any combination thereof. Examples of a network 1030 or network segment 1030 include, but are not limited to, a distributed computing system, a cloud computing system, a wide area network (WAN) (e.g., the Internet, an enterprise network), a local area network (LAN) (e.g., a network associated with an office, a building, a campus or other relatively small geographic space), a telephone network, a direct connection between two computing devices, a peer-to-peer network, and any combinations thereof. A network, such as network 1030, may employ a wired and/or a wireless mode of communication. In general, any network topology may be used.

Information and data can be displayed through a display 1032. Examples of a display 1032 include, but are not limited to, a cathode ray tube (CRT), a liquid crystal display (LCD), a thin film transistor liquid crystal display (TFT-LCD), an organic liquid crystal display (OLED) such as a passive-matrix OLED (PMOLED) or active-matrix OLED (AMOLED) display, a plasma display, and any combinations thereof. The display 1032 can interface to the processor(s) 1001, memory 1003, and fixed storage 1008, as well as other devices, such as input device(s) 1033, via the bus 1040. The display 1032 is linked to the bus 1040 via a video interface 1022, and transport of data between the display 1032 and the bus 1040 can be controlled via the graphics control 1021. In some embodiments, the display is a video projector. In some embodiments, the display is a head-mounted display (HMD) such as a VR headset. In further embodiments, suitable VR headsets include, by way of non-limiting examples, HTC Vive, Oculus Rift, Samsung Gear VR, Microsoft HoloLens, Razer OSVR, FOVE VR, Zeiss VR One, Avegant Glyph, Freefly VR headset, and the like. In still further embodiments, the display is a combination of devices such as those disclosed herein.

In addition to a display 1032, computer system 1031 may include one or more other peripheral output devices 1034 including, but not limited to, an audio speaker, a printer, a storage device, and any combinations thereof. Such peripheral output devices may be connected to the bus 1040 via an output interface 1024. Examples of an output interface 1024 include, but are not limited to, a serial port, a parallel connection, a USB port, a FIREWIRE port, a THUNDERBOLT port, and any combinations thereof.

In addition, or as an alternative, computer system 1031 may provide functionality as a result of logic hardwired or otherwise embodied in a circuit, which may operate in place of or together with software to execute one or more processes or one or more steps of one or more processes described or illustrated herein. Reference to software in this disclosure may encompass logic, and reference to logic may encompass software. Moreover, reference to a computer-readable medium may encompass a circuit (such as an IC) storing software for execution, a circuit embodying logic for execution, or both, where appropriate. The present disclosure encompasses any suitable combination of hardware, software, or both.

Those of skill in the art will appreciate that the various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality.

The various illustrative logical blocks, modules, and circuits described in connection with the embodiments disclosed herein may be implemented or performed with a general-purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

The steps of a method or algorithm described in connection with the embodiments disclosed herein may be embodied directly in hardware, in a software module executed by one or more processor(s), or in a combination of the two. A software module may reside in RAM memory, flash memory, ROM memory, EPROM memory, EEPROM memory, registers, hard disk, a removable disk, a CD-ROM, or any other form of storage medium known in the art. An exemplary storage medium is coupled to the processor such the processor can read information from, and write information to, the storage medium. In the alternative, the storage medium may be integral to the processor. The processor and the storage medium may reside in an ASIC. The ASIC may reside in a user terminal. In the alternative, the processor and the storage medium may reside as discrete components in a user terminal.

In accordance with the description herein, suitable computing devices include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Those of skill in the art will also recognize that select televisions, video players, and digital music players with optional computer network connectivity are suitable for use in the system described herein. Suitable tablet computers, in various embodiments, include those with booklet, slate, and convertible configurations, known to those of skill in the art.

In some embodiments, the computing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smartphone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®. Those of skill in the art will also recognize that suitable media streaming device operating systems include, by way of non-limiting examples, Apple TV®, Roku®, Boxee®, Google TVR, Google Chromecast®, Amazon Fire®, and Samsung® HomeSync®. Those of skill in the art will also recognize that suitable video game console operating systems include, by way of non-limiting examples, Sony® PS3®, Sony® PS4®, Microsoft® Xbox 360®, Microsoft Xbox One, Nintendo® Wii®, Nintendo® Wii U®, and Ouya®.

### Non-Transitory Computer Readable Storage Medium

In some embodiments, the platforms, systems, media, and methods disclosed herein include one or more non-transitory computer readable storage media encoded with a program including instructions executable by the operating system of an optionally networked computing device. In further embodiments, a computer readable storage medium is a tangible component of a computing device. In still further embodiments, a computer readable storage medium is optionally removable from a computing device. In some embodiments, a computer readable storage medium includes, by way of non-limiting examples, CD-ROMs, DVDs, flash memory devices, solid state memory, magnetic disk drives, magnetic tape drives, optical disk drives, distributed computing systems including cloud computing systems and services, and the like. In some cases, the program and instructions are permanently, substantially permanently, semi-permanently, or non-transitorily encoded on the media.

### Computer Program

In some embodiments, the platforms, systems, media, and methods disclosed herein include at least one computer program, or use of the same. A computer program includes a sequence of instructions, executable by one or more processor(s) of the computing device's CPU, written to perform a specified task. Computer readable instructions may be implemented as program modules, such as functions, objects, Application Programming Interfaces (APIs), computing data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Standalone Application

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB.NET, or combinations thereof. Compilation is often performed, at least in part, to create an executable program. In some embodiments, a computer program includes one or more executable complied applications.

### Software Modules

In some embodiments, the platforms, systems, media, and methods disclosed herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on a distributed computing platform such as a cloud computing platform. In some embodiments, software modules are hosted on one or more machines in one location. In other embodiments, software modules are hosted on one or more machines in more than one location.

### Machine Learning

In some embodiments, machine learning algorithms are utilized to adjust the flow of the CSF. In some embodiments, the machine learning algorithms herein employ one or more forms of labels including but not limited to human annotated labels and semi-supervised labels. In some embodiments, the machine learning algorithm utilizes regression modeling, wherein relationships between predictor variables and dependent variables are determined and weighted. In one embodiment, for example, opening pressure of the valve is a dependent variable and is derived from the first measurement, the second measurement, or both.

The human annotated labels can be provided by a hand-crafted heuristic. For example, the hand-crafted heuristic can comprise examining differences between consistent pressure readings and pressure readings with a time-dependent shift. The semi-supervised labels can be determined using a clustering technique to find indicators of shifting pressure measurement and/or disturbances in intracranial pressure similar to those flagged by previous human annotated labels and previous semi-supervised labels. The semi-supervised labels can employ a XGBoost, a neural network, or both.

In some embodiments, a distant supervision method is utilized to adjust flow of the CSF. In some embodiments, the probability that the pressure reading has shifted and/or that an intracranial change is potentially threatening can be constructed on the property is determined using a distant supervision method. The distant supervision method can create a large training set seeded by a small hand-annotated training set. The distant supervision method can comprise positive-unlabeled learning with the training set as the ‘positive’ class. The distant supervision method can employ a logistic regression model, a recurrent neural network, or both. The recurrent neural network can be advantageous for Natural Language Processing (NLP) machine learning.

Examples of machine learning algorithms can include a support vector machine (SVM), a naïve Bayes classification, a random forest, a neural network, deep learning, or other supervised learning algorithm or unsupervised learning algorithm for classification and regression. The machine learning algorithms can be trained using one or more training datasets.

In some embodiments, a machine learning algorithm is used to select catalogue images and recommend project scope. A non-limiting example of a multi-variate linear regression model algorithm is seen below: probability=A₀+A₁(X₁)+A₂(X₂)+A₃(X₃)+A₄(X₄)+A₅(X₅)+A₆(X₆)+A₇(X₇) . . . , wherein Aᵢ(A₁, A₂, A₃, A₄, A₅, A₆, A₇, . . . ) are “weights” or coefficients found during the regression modeling; and Xᵢ(X₁, X₂, X₃, X₄, X₅, X₆, X₇, . . . ) are data collected from the user. Any number of Aᵢ and Xᵢ variable can be included in the model. For example, in a non-limiting example wherein there are 7 Xᵢ terms, X₁ is the number of first measurement readings, X₂ is the number of second measurement readings, and X₃ is the probability that the measurements of the first and/or second sensor is shifting. In some embodiments, the programming language “R” is used to run the model.

In some embodiments, the first machine learning algorithm is trained by a neural network comprising: a first training module creating a first training set comprising a set of accurate pressure measurements and a set of shifting pressure measurements; and a first training module training the neural network using the first training set; a second training module creating a second training set for second stage training comprising the first training set and the pressure measurements incorrectly detected as shifting after the first stage of training; and training the neural network using the second training set.

### Examples of Machine Learning Methodologies

As used in this specification and the appended claims, the terms “artificial intelligence,” “artificial intelligence techniques,” “artificial intelligence operation,” and “artificial intelligence algorithm” generally refer to any system or computational procedure that may take one or more actions that simulate human intelligence processes for enhancing or maximizing a chance of achieving a goal. The term “artificial intelligence” may include “generative modeling,” “machine learning” (ML), or “reinforcement learning” (RL).

As used in this specification and the appended claims, the terms “machine learning,” “machine learning techniques,” “machine learning operation,” and “machine learning model” generally refer to any system or analytical or statistical procedure that may progressively improve computer performance of a task. In some cases, ML may generally involve identifying and recognizing patterns in existing data in order to facilitate making predictions for subsequent data. ML may include a ML model (which may include, for example, a ML algorithm). Machine learning, whether analytical or statistical in nature, may provide deductive or abductive inference based on real or simulated data. The ML model may be a trained model. ML techniques may comprise one or more supervised, semi-supervised, self-supervised, or unsupervised ML techniques. For example, an ML model may be a trained model that is trained through supervised learning (e.g., various parameters are determined as weights or scaling factors). ML may comprise one or more of regression analysis, regularization, classification, dimensionality reduction, ensemble learning, meta learning, association rule learning, cluster analysis, anomaly detection, deep learning, or ultra-deep learning. ML may comprise, but is not limited to: k-means, k-means clustering, k-nearest neighbors, learning vector quantization, linear regression, non-linear regression, least squares regression, partial least squares regression, logistic regression, stepwise regression, multivariate adaptive regression splines, ridge regression, principal component regression, least absolute shrinkage and selection operation (LASSO), least angle regression, canonical correlation analysis, factor analysis, independent component analysis, linear discriminant analysis, multidimensional scaling, non-negative matrix factorization, principal components analysis, principal coordinates analysis, projection pursuit, Sammon mapping, t-distributed stochastic neighbor embedding, AdaBoosting, boosting, gradient boosting, bootstrap aggregation, ensemble averaging, decision trees, conditional decision trees, boosted decision trees, gradient boosted decision trees, random forests, stacked generalization, Bayesian networks, Bayesian belief networks, naïve Bayes, Gaussian naïve Bayes, multinomial naïve Bayes, hidden Markov models, hierarchical hidden Markov models, support vector machines, encoders, decoders, auto-encoders, stacked auto-encoders, perceptrons, multi-layer perceptrons, artificial neural networks, feedforward neural networks, convolutional neural networks, recurrent neural networks, long short-term memory, deep belief networks, deep Boltzmann machines, deep convolutional neural networks, deep recurrent neural networks, or generative adversarial networks.

Methods and/or systems of the disclosure can process, analyze, and/or classify the status of the subject, as described elsewhere herein. In some cases, the processing, analyzing, and/or classifying of the flow rate of the implantable system and/or one or more features of the status of the subject may be conducted by way of one or more machine learning algorithms and/or one or more predictive models with instructions provided with one or more processors as disclosed herein. For example, one or more machine learning algorithms and/or predictive models may process one or more, or two or more features of the valve opening rate as described elsewhere herein.

In some cases, the subject's and/or plurality of subjects' phenotypes may be determined and/or predicted with one or more machine learning algorithms and/or one or more predictive models with a sensitivity of at least about 70%, at least about 75%, at least about 80%, at least about 85% or at least about 90%.

In some cases, the status of the subject may be determined and/or predicted with one or more machine learning algorithms and/or one or more predictive models with a sensitivity of up to about 70%, up to about 75%, up to about 80%, up to about 85% or up to about 90%.

In some cases, the status of the subject may be determined and/or predicted with one or more machine learning algorithms and/or one or more predictive models with a specificity of at least about 70%, at least about 75%, at least about 80%, at least about 85% or at least about 90%.

In some cases, the status of the subject may be determined and/or predicted with one or more machine learning algorithms and/or one or more predictive models with a specificity of up to about 70%, up to about 75%, up to about 80%, up to about 85% or up to about 90%.

In some cases, the status of the subject may be determined and/or predicted with one or more machine learning algorithms and/or one or more predictive models with a positive predictive value of at least about 70%, at least about 75%, at least about 80%, at least about 85% or at least about 90%.

In some cases, the status of the subject may be determined and/or predicted with one or more machine learning algorithms and/or one or more predictive models with a positive predictive value of up to about 70%, up to about 75%, up to about 80%, up to about 85% or up to about 90%.

In some cases, the status of the subject may be determined and/or predicted with one or more machine learning algorithms and/or one or more predictive models with a negative predictive value of at least about 70%, at least about 75%, at least about 80%, at least about 85% or at least about 90%.

In some cases, the status of the subject may be determined and/or predicted with one or more machine learning algorithms and/or one or more predictive models with a negative predictive value of up to about 70%, up to about 75%, up to about 80%, up to about 85% or up to about 90%.

In some cases, the status of the subject may be determined and/or predicted with one or more machine learning algorithms and/or one or more predictive models with an Area Under the Receiver Operating Characteristic Curve (AUROC) of at least about 0.65, at least about 0.70, at least about 0.75, at least about 0.80, at least about 0.82, at least about 0.84, at least about 0.86, at least about 0.88, or at least about 0.90.

In some cases, the status of the subject may be determined and/or predicted with one or more machine learning algorithms and/or one or more predictive models with an Area Under the Receiver Operating Characteristic (AUROC) of up to about 0.65, up to about 0.70, up to about 0.75 up to about 0.80, up to about 0.82, up to about 0.84, up to about 0.86 up to about 0.88, or up to about 0.90.

An algorithm and/or predictive model can be implemented by way of software upon execution by central processing unit. In some cases, the predictive model may comprise a machine learning predictive model. In some cases, the machine learning predictive model may comprise one or more statistical, machine learning, or artificial intelligence algorithms. Examples of utilized algorithms, machine learning algorithms, and/or predictive models may include a support vector machine (SVM), a naïve Bayes classification, a random forest, a neural network (such as a deep neural network (DNN)), a recurrent neural network (RNN), a deep RNN, a long short-term memory (LSTM) recurrent neural network (RNN), decision tree algorithm, unsupervised clustering algorithm, a supervised clustering algorithm, unsupervised clustering algorithm, a regression algorithm, a gradient-boosting algorithm (e.g., a gradient-boosting implementation of a machine learning algorithm and/or predictive model such as a gradient-boosted decision trees), a gated recurrent unit (GRU), supervised learning algorithm, unsupervised learning algorithm, statistical, deep-learning algorithm for classification and regression, or any combination thereof. In some cases, the recurrent neural network may comprise units which can be LSTM units or GRU. In some cases, the predictive model and/or the machine learning algorithm may comprise an ensemble of one or more predictive models and/or machine learning algorithms.

The machine learning predictive model may likewise involve the estimation of ensemble models, comprised of multiple predictive models, and utilize techniques such as gradient boosting, for example in the construction of gradient-boosting decision trees. The machine learning predictive model may be trained using one or more training datasets corresponding to flow rates of the CSF.

Training records may be constructed from sequences of observations. Such sequences may comprise a fixed length for ease of data processing. For example, sequences may be zero-padded or selected as independent subsets of a single subject's records.

The one or more predictive models and/or one or more machine learning algorithms may process one or more input features to generate one or more output values comprising the predicted status of the subject. For example, such prediction of the status of the subject may comprise a binary classification of a healthy/normal health state (e.g., absence of a disease or disorder) or an adverse health state (e.g., presence of a disease or disorder), a classification between a group of categorical labels (e.g., ‘no disease or disorder’, ‘apparent disease or disorder’, and ‘likely disease or disorder’), a likelihood (e.g., relative likelihood or probability) of developing a particular disease or disorder, a score indicative of a presence of disease or disorder, a score indicative of a level of systemic inflammation experienced by the patient, a ‘risk factor’ for the likelihood of mortality of the patient, a prediction of the time at which the patient is expected to have developed the disease or disorder, a confidence interval for any numeric predictions, or any combination thereof. Various predictive model and/or machine learning algorithms may be cascaded such that the output of one or more predictive models and/or one or more machine learning algorithms may be used as one or more input features to subsequent layers or subsections of the one or more predictive model and/or one or more machine learning algorithms.

In order to train the one or more predictive models and/or the one or more machine learning algorithms (e.g., by determining weights and correlations of the predictive model and/or the machine learning algorithm) to generate real-time classifications or predictions, the model can be trained using datasets (e.g., training datasets), described elsewhere herein. Such datasets may be sufficiently large to generate statistically significant classifications or predictions. For example, datasets may comprise databases of de-identified data including one or more molecular signatures, other measurements from a hospital or other clinical setting, or any combination thereof.

Datasets, as described elsewhere herein, may be split into subsets (e.g., discrete or overlapping), such as a training dataset, a development dataset, and a test dataset. For example, a dataset may be split into a training dataset comprising 80% of the dataset, a development dataset comprising 10% of the dataset, and a test dataset comprising 10% of the dataset. The training dataset may comprise about 10%, about 20%, about 30%, about 40%, about 50%, about 60%, about 70%, about 80%, or about 90% of the dataset. The development dataset may comprise about 10%, about 20%, about 30%, about 40%, about 50%, about 60%, about 70%, about 80%, or about 90% of the dataset. The test dataset may comprise about 10%, about 20%, about 30%, about 40%, about 50%, about 60%, about 70%, about 80%, or about 90% of the dataset. Training sets (e.g., training datasets) may be selected by random sampling of a set of data corresponding to one or more user cohorts to ensure independence of sampling. In some cases, training sets (e.g., training datasets) may be selected by proportionate sampling of a set of data corresponding to one or more user cohorts to ensure independence of sampling.

To improve the accuracy of predictive model and/or machine learning algorithm predictions and reduce overfitting of the predictive model and/or machine learning algorithm, the datasets may be augmented to increase the number of samples within the training set. For example, data augmentation may comprise rearranging the order of observations in a training record. To accommodate datasets having missing observations, methods to impute missing data may be used, such as forward-filling, back-filling, linear interpolation, and multi-task Gaussian processes. Datasets may be filtered to remove confounding factors. For example, within a database, a subset of users may be excluded.

Neural network techniques, such as dropout or regularization, may be used during training the one or more predictive models and/or one or more machine learning algorithms to prevent overfitting. The neural network may comprise a plurality of sub-networks, each of which is configured to generate a classification or prediction of a different type of output information (e.g., which may be combined to form an overall output of the neural network). The one or more predictive models and/or the one or more machine learning algorithms may alternatively utilize statistical or related algorithms including random forest, classification and regression trees, support vector machines, discriminant analyses, regression techniques, ensemble and gradient-boosted variations thereof, or any combination thereof.

When the one or more predictive models and/or the one or more machine learning algorithms generate a classification or a prediction of parameters needed to predict the status of the subject, a notification (e.g., alert or alarm) may be generated and transmitted to a health care provider, such as a physician, nurse, health care personnel managing, or any combination thereof, treating a user e.g., a subject within a hospital. Notifications may be transmitted via an automated phone call, a short message service (SMS), multimedia message service (MMS) message, an e-mail, an alert within a dashboard, or any combination thereof. The notification may comprise output information such as a prediction of the status of the subject.

To validate the performance of the one or more predictive models and/or one more machine learning algorithms, different performance metrics may be generated. For example, an area under the receiver-operating curve (AUROC) may be used to determine the diagnostic and/or classification capability of the one or more predictive models and/or one or more machine learning algorithms. For example, the one or more predictive models and/or one or more machine learning algorithms may use classification thresholds which are adjustable, such that specificity and sensitivity are tunable, and the receiver-operating characteristic curve (ROC) can be used to identify the different operating points corresponding to different values of specificity and sensitivity of the one or more predictive models and/or one or more machine learning algorithms.

In some cases, such as when datasets are not sufficiently large, cross-validation may be performed to assess the robustness of one or more predictive models and/or one or more machine learning algorithms across different training and testing datasets.

To calculate performance metrics such as sensitivity, specificity, accuracy, positive predictive value (PPV), negative predictive value (NPV), AUPRC, AUROC, any combination thereof, or similar, the following definitions may be used. A “false positive” may refer to an outcome in which a positive outcome or result has been incorrectly or prematurely generated. A “true positive” may refer to an outcome in which positive outcome or result has been correctly generated. A “false negative” may refer to an outcome in which a negative outcome or result has been generated. A “true negative” may refer to an outcome in which a negative outcome or result has been generated.

The one or more predictive models and/or one or more machine learning algorithms may be trained until certain pre-determined conditions for accuracy or performance are satisfied, such as having minimum desired values corresponding to classification and/or diagnostic accuracy measures. For example, the diagnostic accuracy measure may correspond to prediction of a likelihood of occurrence of a specific status of a subject. Examples of diagnostic accuracy measures may include sensitivity, specificity, positive predictive value (PPV), negative predictive value (NPV), accuracy, area under the precision-recall curve (AUPRC), and area under the curve (AUC) of a Receiver Operating Characteristic (ROC) curve (AUROC) corresponding to the diagnostic accuracy of detecting or predicting a phenotype.

For example, such a pre-determined condition may be that the sensitivity of predicting the status of the subject comprises a value of, for example, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

As another example, such a pre-determined condition may be that the specificity of predicting the status of the subject comprises a value of, for example, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

As another example, such a pre-determined condition may be that the positive predictive value (PPV) of predicting the status of the subject comprises a value of, for example, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

As another example, such a pre-determined condition may be that the negative predictive value (NPV) of predicting the status of the subject comprises a value of, for example, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

As another example, such a pre-determined condition may be that the area under the curve (AUC) of a Receiver Operating Characteristic (ROC) curve (AUROC) of predicting the status of the subject comprises a value of at least about 0.50, at least about 0.55, at least about 0.60, at least about 0.65, at least about 0.70, at least about 0.75, at least about 0.80, at least about 0.85, at least about 0.90, at least about 0.95, at least about 0.96, at least about 0.97, at least about 0.98, or at least about 0.99.

As another example, such a pre-determined condition may be that the area under the precision-recall curve (AUPRC) of predicting the status of the subject comprises a value of at least about 0.10, at least about 0.15, at least about 0.20, at least about 0.25, at least about 0.30, at least about 0.35, at least about 0.40, at least about 0.45, at least about 0.50, at least about 0.55, at least about 0.60, at least about 0.65, at least about 0.70, at least about 0.75, at least about 0.80, at least about 0.85, at least about 0.90, at least about 0.95, at least about 0.96, at least about 0.97, at least about 0.98, or at least about 0.99.

In some embodiments, the trained model may be trained or configured to predict the status of the subject with a sensitivity of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

In some embodiments, the trained model may be trained or configured to predict the status of the subject with a specificity of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

In some embodiments, the trained model may be trained or configured to predict the status of the subject with a positive predictive value (PPV) of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

In some embodiments, the trained model may be trained or configured to predict the status of the subject with a negative predictive value (NPV) of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

In some embodiments, the trained model may be trained or configured to predict the status of the subject with an area under the curve (AUC) of a Receiver Operating Characteristic (ROC) curve (AUROC) of at least about 0.50, at least about 0.55, at least about 0.60, at least about 0.65, at least about 0.70, at least about 0.75, at least about 0.80, at least about 0.85, at least about 0.90, at least about 0.95, at least about 0.96, at least about 0.97, at least about 0.98, or at least about 0.99.

In some embodiments, the trained model may be trained or configured to predict the status of the subject with an area under the precision-recall curve (AUPRC) of at least about 0.10, at least about 0.15, at least about 0.20, at least about 0.25, at least about 0.30, at least about 0.35, at least about 0.40, at least about 0.45, at least about 0.50, at least about 0.55, at least about 0.60, at least about 0.65, at least about 0.70, at least about 0.75, at least about 0.80, at least about 0.85, at least about 0.90, at least about 0.95, at least about 0.96, at least about 0.97, at least about 0.98, or at least about 0.99.

In some embodiments, the machine learning analysis is performed by a device executing one or more programs (e.g., one or more programs stored in the Non-Persistent Memory or in the Persistent Memory) including instructions to perform the data analysis. In some embodiments, the data analysis is performed by a system comprising at least one processor (e.g., the processing core) and memory (e.g., one or more programs stored in the Non-Persistent Memory or in the Persistent Memory) comprising instructions to perform the data analysis.

Training the ML model may include, in some cases, selecting one or more untrained data models to train using a training data set. The selected untrained data models may include any type of untrained ML models for supervised, semi-supervised, self-supervised, or unsupervised machine learning. The selected untrained data models may be specified based upon input (e.g., user input) specifying relevant parameters to use as predicted variables or other variables to use as potential explanatory variables. For example, the selected untrained data models may be specified to generate an output (e.g., a prediction) based upon the input. Conditions for training the ML model from the selected untrained data models may likewise be selected, such as limits on the ML model complexity or limits on the ML model refinement past a certain point. The ML model may be trained (e.g., via a computer system such as a server) using the training data set. In some cases, a first subset of the training data set may be selected to train the ML model. The selected untrained data models may then be trained on the first subset of training data set using appropriate ML techniques, based upon the type of ML model selected and any conditions specified for training the ML model. In some cases, due to the processing power requirements of training the ML model, the selected untrained data models may be trained using additional computing resources (e.g., cloud computing resources). Such training may continue, in some cases, until at least one aspect of the ML model is validated and meets selection criteria to be used as a predictive model.

In some cases, one or more aspects of the ML model may be validated using a second subset of the training data set (e.g., distinct from the first subset of the training data set) to determine accuracy and robustness of the ML model. Such validation may include applying the ML model to the second subset of the training data set to make predictions derived from the second subset of the training data. The ML model may then be evaluated to determine whether performance is sufficient based upon the derived predictions. The sufficiency criteria applied to the ML model may vary depending upon the size of the training data set available for training, the performance of previous iterations of trained models, or user-specified performance requirements. If the ML model does not achieve sufficient performance, additional training may be performed. Additional training may include refinement of the ML model or retraining on a different first subset of the training dataset, after which the new ML model may again be validated and assessed. When the ML model has achieved sufficient performance, in some cases, the ML may be stored for present or future use. The ML model may be stored as sets of parameter values or weights for analysis of further input (e.g., further relevant parameters to use as further predicted variables, further explanatory variables, further user interaction data, etc.), which may also include analysis logic or indications of model validity in some instances. In some cases, a plurality of ML models may be stored for generating predictions under different sets of input data conditions. In some embodiments, the ML model may be stored in a database (e.g., associated with a server).

In some embodiments, the one or more predictive models and/or the one or more machine learning algorithms may comprise a neural network or a convolutional neural network. See, Vincent et al., 2010, “Stacked denoising autoencoders: Learning useful representations in a deep network with a local denoising criterion,” J Mach Learn Res 11, pp. 3371-3408; Larochelle et al., 2009, “Exploring strategies for training deep neural networks,” J Mach Learn Res 10, pp. 1-40; and Hassoun, 1995, Fundamentals of Artificial Neural Networks, Massachusetts Institute of Technology, each of which is hereby incorporated by reference.

Independent component analysis (ICA), described elsewhere herein, in the unsupervised dimensionality-reduction of molecular signatures, is described in Lee, T.-W. (1998): Independent component analysis: Theory and applications, Boston, Mass: Kluwer Academic Publishers, ISBN 0-7923-8261-7, and Hyvärinen, A.; Karhunen, J.; Oja, E. (2001): Independent Component Analysis, New York: Wiley, ISBN 978-0-471-40540-5, which is hereby incorporated by reference in its entirety.

Principal component analysis (PCA), described elsewhere herein, in the unsupervised dimensionality-reduction of moleculr signatures, is described in Jolliffe, I. T. (2002). Principal Component Analysis. Springer Series in Statistics. New York: Springer-Verlag. doi: 10.1007/b98835. ISBN 978-0-387-95442-4, which is hereby incorporated by reference in its entirety.

SVMs are described in Cristianini and Shawe-Taylor, 2000, “An Introduction to Support Vector Machines,” Cambridge University Press, Cambridge; Boser et al., 1992, “A training algorithm for optimal margin classifiers,” in Proceedings of the 5^{th} Annual ACM Workshop on Computational Learning Theory, ACM Press, Pittsburgh, Pa., pp. 142-152; Vapnik, 1998, Statistical Learning Theory, Wiley, New York; Mount, 2001, Bioinformatics: sequence and genome analysis, Cold Spring Harbor Laboratory Press, Cold Spring Harbor, N.Y.; Duda, Pattern Classification, Second Edition, 2001, John Wiley & Sons, Inc., pp. 259, 262-265; and Hastie, 2001, The Elements of Statistical Learning, Springer, New York; and Furey et al., 2000, Bioinformatics 16, 906-914, each of which is hereby incorporated by reference in its entirety. When used for classification, SVMs separate a given set of binary labeled data with a hyper-plane that is maximally distant from the labeled data. For cases in which no linear separation is possible, SVMs can work in combination with the technique of ‘kernels’, which automatically realizes a non-linear mapping to a feature space. The hyper-plane found by the SVM in feature space corresponds to a non-linear decision boundary in the input space.

Decision trees are described generally by Duda, 2001, Pattern Classification, John Wiley & Sons, Inc., New York, pp. 395-396, which is hereby incorporated by reference. Tree-based methods partition the feature space into a set of rectangles, and then fit a model (like a constant) in each one. In some embodiments, the decision tree is random forest regression. One specific algorithm that can be used is a classification and regression tree (CART). Other specific decision tree algorithms include, but are not limited to, ID3, C4.5, MART, and Random Forests. CART, ID3, and C4.5 are described in Duda, 2001, Pattern Classification, John Wiley & Sons, Inc., New York. pp. 396-408 and pp. 411-412, which is hereby incorporated by reference. CART, MART, and C4.5 are described in Hastie et al., 2001, The Elements of Statistical Learning, Springer-Verlag, New York, Chapter 9, which is hereby incorporated by reference in its entirety. Random Forests are described in Breiman, 1999, “Random Forests—Random Features,” Technical Report 567, Statistics Department, U.C. Berkeley, September 1999, which is hereby incorporated by reference in its entirety.

Clustering (e.g., unsupervised clustering model algorithms and supervised clustering model algorithms) is described at pages 211-256 of Duda and Hart, Pattern Classification and Scene Analysis, 1973, John Wiley & Sons, Inc., New York, (hereinafter “Duda 1973”) which is hereby incorporated by reference in its entirety. As described in Section 6.7 of Duda 1973, the clustering problem may be described as one of finding natural groupings in a dataset. To identify natural groupings, two issues are addressed. First, a way to measure similarity (or dissimilarity) between two samples is determined. This metric (similarity measure) may be used to ensure that the samples in one cluster are more like one another than they are to samples in other clusters. Second, a mechanism for partitioning the data into clusters using the similarity measure may be determined. Similarity measures are discussed in Section 6.7 of Duda 1973, where it is stated that one way to begin a clustering investigation may be to define a distance function and to compute the matrix of distances between all pairs of samples in the training set. If distance is a good measure of similarity, then the distance between reference entities in the same cluster may be significantly less than the distance between the reference entities in different clusters. However, as stated on page 215 of Duda 1973, clustering may not require the use of a distance metric. For example, a nonmetric similarity function s (x, x′) can be used to compare two vectors x and x′. Conventionally, s (x, x′) is a symmetric function whose value may be large when x and x′ are somehow “similar.” An example of a nonmetric similarity function s (x, x′) is provided on page 218 of Duda 1973. Once a method for measuring “similarity” or “dissimilarity” between points in a dataset has been selected, clustering may require a criterion function that measures the clustering quality of any partition of the data. Partitions of the data set that extremize the criterion function may be used to cluster the data. See page 217 of Duda 1973. Criterion functions are discussed in Section 6.8 of Duda 1973. More recently, Duda et al., Pattern Classification, 2^{nd} edition, John Wiley & Sons, Inc. New York, has been published. Pages 537-563 describe clustering in detail. More information on clustering techniques can be found in Kaufman and Rousseeuw, 1990, Finding Groups in Data: An Introduction to Cluster Analysis, Wiley, New York, N.Y.; Everitt, 1993, Cluster analysis (3d ed.), Wiley, New York, N.Y.; and Backer, 1995, Computer-Assisted Reasoning in Cluster Analysis, Prentice Hall, Upper Saddle River, New Jersey, each of which is hereby incorporated by reference. Particular exemplary clustering techniques that can be used in the present disclosure include, but are not limited to, hierarchical clustering (agglomerative clustering using nearest-neighbor algorithm, farthest-neighbor algorithm, the average linkage algorithm, the centroid algorithm, or the sum-of-squares algorithm), k-means clustering, fuzzy k-means clustering algorithm, Jarvis-Patrick clustering, or any combination thereof. In some embodiments, the clustering comprises unsupervised clustering, where no preconceived notion of what clusters should form when the training set is clustered, are imposed.

Regression models, such as that of the multi-category logit models, are described in Agresti, An Introduction to Categorical Data Analysis, 1996, John Wiley & Sons, Inc., New York, Chapter 8, which is hereby incorporated by reference in its entirety. In some embodiments, the one or more predictive model and/or one or more machine learning algorithms may make use of a regression model disclosed in Hastie et al., 2001, The Elements of Statistical Learning, Springer-Verlag, New York, which is hereby incorporated by reference in its entirety. In some embodiments, gradient-boosting models may be used toward, for example, the classification algorithms described herein; these gradient-boosting models are described in Boehmke, Bradley; Greenwell, Brandon (2019). “Gradient Boosting”. Hands-On Machine Learning with R. Chapman & Hall. pp. 221-245. ISBN 978-1-138-49568-5., which is hereby incorporated by reference in its entirety. In some embodiments, ensemble modeling techniques may be used, for example, toward the classification algorithms described herein; these ensemble modeling techniques are described in the implementation of classification models herein, are described in Zhou Zhihua (2012). Ensemble Methods: Foundations and Algorithms. Chapman and Hall/CRC. ISBN 978-1-439-83003-1, which is hereby incorporated by reference in its entirety.

One aspect provided herein is an implantable system for monitoring intracranial pressure (ICP), the system comprising: a first sensor configured to measure the absolute ICP of the user and output a first measurement; a second sensor configured to measure a fixed air pressure within a hermetically sealed compartment and output a second measurement; a controller configured to calculate a drift corrected absolute ICP based at least in part on a difference between the first and second measurements; and a shunt wireless communication device configured to transmit the absolute ICP.

In some embodiments, the shunt wireless communication device receives the atmospheric pressure from an external wand device and is configured to calculate the ICP based at least in part as the difference between the first, second, and third measurements, i.e., first minus second minus third measurements.

In some embodiments, the first measurement includes the absolute ICP, namely ICP plus atmospheric pressure, output from a first pressure sensor. In some embodiments, the ICP measurement may include the ICP, atmospheric pressure, and sensor drift, the second measurement may include an air pressure and sensor drift, and the third measurement may include the atmospheric pressure.

In some embodiments, the second measurement is output by a second pressure sensor that is positioned outside the fluid column in a hermetically sealed cavity filled with air, which is at an initial atmospheric pressure (P₀). The second measurement includes P₀ and sensor drift. Because the sealed cavity is rigid and not vented to the atmosphere, that initial pressure, P₀, must be removed through calibration. Because P₀ is known at time of manufacture, its value is stored in non-volatile memory (NV) and is used to subtract it from the second measurement. This constitutes calibrating for P₀.

In some embodiments, the valve, the first pressure sensor, and the flow rate sensor are integrated.

In some embodiments, the valve, the first pressure sensor, the second pressure sensor, and the flow rate sensor are integrated.

In some embodiments, the first pressure sensor, the second pressure sensor, and the flow rate sensor are integrated and proximal to, but separate from, the valve.

In some embodiments, the controller is further configured to adjust the opening pressure threshold of the valve or to determine possible shunt malfunction or occlusion based on an algorithm, that includes some or all of the ICP waveform, mean ICP, atmospheric pressure waveform, mean atmospheric pressure, flow rate waveform, mean flow rate, one or more of a machine learning model, artificial intelligence, and signal processing techniques.

In some embodiments, the system may be configured to determine a status of the user based on the absolute ICP, ICP, measured flow rate, or any combination thereof. The controller may be configured to adjust the opening pressure threshold of the valve based on the status and may provide textual feedback, including clinical messages, to the patient through an app on the external wand device, which is typically a smartphone. In some embodiments, the controller may be further configured to adjust the opening pressure threshold of the valve based at least in part on the ICP, the measured flow rate, or any combination thereof.

In some embodiments, the compartment may comprise the first pressure sensor, the flow rate sensor, the accelerometer, a second pressure sensor and a wireless charging receiver.

In some embodiments, the third pressure sensor is inside the external wand device, typically a smartphone.

### Terms and Definitions

Unless otherwise defined, all technical terms used herein have the same meaning as commonly understood by one of ordinary skill in the art to which this disclosure belongs.

As used herein, the singular forms “a,” “an,” and “the” include plural references unless the context clearly dictates otherwise. Any reference to “or” herein is intended to encompass “and/or” unless otherwise stated.

As used herein, the term “about” in some cases refers to an amount that is approximately the stated amount.

As used herein, the term “about” refers to an amount that is near the stated amount by 10%, 5%, or 1%, including increments therein.

As used herein, the term “about” in reference to a percentage refers to an amount that is greater or less the stated percentage by 10%, 5%, or 1%, including increments therein.

As used herein, the phrases “at least one,” “one or more,” and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

Whenever the term “at least,” “greater than” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

### EXAMPLES

The following illustrative examples are representative of embodiments of the software applications, systems, and methods described herein and are not meant to be limiting in any way.

### Example 1—Autonomous Mode Monitoring

Below is an exemplary pseudocode performed by a controller of a shunt system in an autonomous mode:

| | |
|---|---|
| DP_thresh_H = <DP high threshold> | // this value comes from learning |

algorithm

| | |
|---|---|
| DP_thresh_L = <DP low threshold> | // this value comes from learning |

algorithm

| | |
|---|---|
| ICP = P1−p2 | // microcontroller result from sensor |

data

| | |
|---|---|
| T = 4 hours | // further adjustments not allowed for T hours |
| Monitor = Yes | // continuous monitoring in closed-loop mode |

Do {
IF timer = 0 THEN

| | |
|---|---|
| IF ICP > DP_thresh_H THEN | // pressure is too high |
| DP_thresh = DP_thresh−1 | //decrements threshold |

Timer = T

| | |
|---|---|
| ElseIF ICP < DP_thresh_H THEN | // pressure is too low |
| DP_thresh = DP_thresh+1 | //increments threshold |

```
    Timer = T
  End IF
END IF
}
While monitor = yes
// An interrupt from the external wand device is needed to set monitor = NO, to exit
loop
```

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure.

What is claimed is:

1. An implantable system for monitoring intracranial pressure (ICP) in a subject, the system comprising:
(a) a conduit for the flow of cerebrospinal fluid (CSF);
(b) a hermetically sealed compartment;
(c) a first pressure sensor configured to measure an absolute ICP and output a measurement, wherein the first pressure sensor is hermetically sealed in the compartment, and wherein the first pressure sensor comprises a surface exposed to the CSF in the conduit;
(d) a microcontroller disposed in the hermetically sealed compartment that receives multiple inputs including at least the output of the first pressure sensor; and
(e) an accelerometer disposed in the hermetically sealed compartment for monitoring a subject's positional status, and wherein the accelerometer communicates an output to the microcontroller.

2. An implantable system for monitoring intracranial pressure (ICP) in a subject, the system comprising:
(a) a conduit for the flow of cerebrospinal fluid (CSF);
(b) a hermetically sealed compartment;
(c) a flow rate sensor disposed in the hermetically sealed compartment, mounted to an outer surface of the CSF conduit such that the sensor is hermetically isolated from the CSF, and wherein the flow rate sensor outputs a measurement; and
(d) a microcontroller disposed in the hermetically sealed compartment that receives a plurality of inputs including at least the output of the flow rate sensor; and
(e) an accelerometer disposed in the hermetically sealed compartment for monitoring a subject's positional status and to communicate an output to the microcontroller.

3. The implantable system of any one of the preceding claims, wherein the hermetically sealed compartment comprises a wireless charging circuit configured to be charged by an external charger.

4. The implantable system of any one of the preceding claims, wherein the implantable system and a ventriculoperitoneal (VP) shunt valve are integrated.

5. The implantable system of any one of the preceding claims, wherein the implantable system is proximal and connected to a VP shunt valve.

6. The implantable system of any one of the preceding claims, wherein electronic components of the implantable system are mounted on a printed circuit board (PCB) within the hermetically sealed compartment.

7. The implantable system of any one of the preceding claims, wherein the absolute ICP is the sum of ICP and atmospheric pressure.

8. The implantable system of any one of the preceding claims, further comprising a circuit configured to measure a common mode output voltage of the first pressure sensor, wherein the common mode output voltage is used to measure the subject's body temperature, and wherein the common mode output voltage is linearly and precisely related to temperature: T=aV+b, wherein a and b are constants.

9. The implantable system of any one of the preceding claims, wherein a drift in pressure measurement of the first pressure sensor caused by temperature is corrected from the temperature measurement obtained from the common mode voltage of the first pressure sensor.

10. The implantable system of any one of the preceding claims, wherein the microcontroller is further configured to:
(a) prior to pressure measurements, turn on a switch to sample a common mode output voltage from the first pressure sensor;
(b) store the common mode output voltage; and
(c) turn off the switch to output differential pressure measurements.

11. The implantable system of any one of the preceding claims, wherein the ICP=m*V_diff+b+TC*delta_Temp; TC=temp coeff, where m is a first constant, V_diff is the differential output of the first pressure sensor, b is a second constant, TC is a temperature coefficient, and delta_Temp is a change in temperature.

12. The implantable system of any one of the preceding claims, wherein the wireless charging circuit uses near field communication (NFC).

13. The implantable system of any one of the preceding claims, wherein the hermetically sealed compartment further comprises a wireless modem configured to receive and transmit data between the microcontroller and an external device.

14. The implantable system of any one of the preceding claims, wherein the hermetically sealed compartment further comprises an antenna for receiving and transmitting wireless signals residing within the hermetically sealed compartment.

15. The implantable system of any one of the preceding claims, wherein the drift in pressure measurement of the first pressure sensor is configured to be corrected by:
(a) storing an initial pressure of the air enclosed within the hermetically sealed compartment during assembly in non-volatile memory in the microcontroller;
(b) measuring a second pressure of the air in the hermetically sealed compartment using a second pressure sensor that comprises a membrane or surface exposed to the air within the compartment and not in contact with the CSF, wherein the second pressure sensor is configured to measure an atmospheric pressure within the compartment and output a second measurement;
(c) calculating a difference between the initial pressure and the second measurement to determine a sensor drift; and
(d) calculating a drift-corrected absolute ICP based at least in part on a difference between a first measurement ICP and the sensor drift, wherein the first measurement ICP is produced by the first pressure sensor.

16. The implantable system of any one of the preceding claims, wherein the microcontroller is further configured to receive the atmospheric pressure from an external device.

17. The implantable system of any one of the preceding claims, wherein the microcontroller is further configured to compute the ICP based at least in part on a difference between the absolute ICP and the atmospheric pressure.

18. The implantable system of any one of the preceding claims, wherein the microcontroller is configured to compute the ICP based at least in part on a difference between the drift-corrected absolute ICP and the atmospheric pressure.

19. The implantable system of any one of the preceding claims, wherein the external device comprises a smartphone or a tablet computer, wherein the microcontroller is further configured to transmit a plurality of measurements output by an analog-to-digital converter (ADC) to the external device, and wherein the ICP is calculated by an internal processor digital signal processor (DSP) in the external device.

20. The implantable system of any one of the preceding claims, wherein a subject status includes a message to be displayed on a graphical user interface of the external device including a diagnostic summary of ICP health, flow rate health, or recommendation for adjustment to flow resistance of the VP shunt valve, or any combination thereof.

21. The implantable system of any one of the preceding claims, wherein the microcontroller is further configured to send a message to the external device for display on the graphical user interface.

22. The implantable system of any one of the preceding claims, wherein the external device is connected to a cloud network.

23. The implantable system of any one of the preceding claims, wherein the CSF flowing out of the conduit is directly input to a VP shunt.

24. The implantable system of any one of the preceding claims, wherein a subject positional status includes at least: sitting, standing, or laying down.

25. The implantable system of any one of the preceding claims, wherein the microcontroller is configured to exclude pressure measurements if the accelerometer indicates subject movement during the measurement, wherein data is excluded upon detection of motion, and wherein data will begin to be captured again if two conditions are met:
(a) a programmable timer expires, and
(b) no motion of the subject is detected.

26. The implantable system of any one of the preceding claims, wherein the threshold for motion detection is increased to allow for light activity.

27. The implantable system of any one of the preceding claims, wherein a lowpass filter time constant is applied to the ICP waveform to reduce variation resulting from a motion detection threshold.

28. The implantable system of any one of the preceding claims, wherein the microcontroller is further configured to transmit a change in subject position to the external device if the accelerometer detects a change in the subject positional status from laying to sitting, or from sitting to laying, sitting to standing, laying to standing, or any combination thereof.

29. The implantable system of any one of the preceding claims, wherein the implantable system further comprises a motor.

30. The implantable system of any one of the preceding claims, wherein the microcontroller is further configured to control the motor to adjust the flow resistance of the VP shunt valve by one or more increments.

31. The implantable system of any one of the preceding claims, wherein the microcontroller is further configured to compensate for increase in CSF flow upon standing or decrease in CSF flow upon laying down, based on input from the accelerometer, by commanding a motor to adjust the flow resistance of the VP shunt valve by one or more increments.

32. The implantable system of any one of the preceding claims, further comprising a valve, wherein the valve comprises one or more of a ball-spring valve, a floating ball valve, a trunnion mounted ball valve, a top entry ball valve, a side entry ball valve, a three-way ball valve, a ball check valve, a flapper check valve, a disc check valve, a ball relief valve, a spring safety valve, or a spring-loaded pressure relief valve.

33. The implantable system of any one of the preceding claims, wherein the implantable system further comprises the controller and a shunt wireless communication device.

34. The implantable system of any one of the preceding claims, wherein the valve, the flow rate sensor, the first pressure sensor, the shunt wireless communication device, the controller, or any combination thereof is magnetic or non-magnetic.

35. The implantable system of any one of the preceding claims, wherein an opening pressure threshold of the valve is manually adjustable.

36. The implantable system of any one of the preceding claims, wherein the opening pressure threshold of the valve is manually adjustable by an external magnetic device.

37. The implantable system of any one of the preceding claims, further comprising a motor configured to adjust an opening pressure threshold of the valve.

38. The implantable system of any one of the preceding claims, wherein the controller is further configured to direct the motor to adjust the opening pressure threshold.

39. The implantable system of any one of the preceding claims, wherein the shunt wireless communication device is further configured to receive a target opening pressure threshold, and wherein the controller is further configured to control the motor based at least in part on the target opening pressure threshold.

40. The implantable system of any one of the preceding claims, wherein a sensor circuit detects a motor position and the controller configures the modem to transmit this position to an external wand device.

41. The implantable system of any one of the preceding claims, wherein the controller is further configured to adjust the opening pressure threshold of the valve based at least in part on the ICP, the first measurement, the second measurement, a third measurement, or any combination thereof.

42. The implantable system of any one of the preceding claims, wherein the controller is further configured to receive a motor position from a position sensor, and wherein the shunt wireless communication device is further configured to transmit an opening pressure threshold confirmation based on the motor position.

43. The implantable system of any one of the preceding claims, further comprising a memory configured to store the first measurement, second measurement, ICP, sensor drift, target ICP, or any combination thereof.

44. The implantable system of any one of the preceding claims, wherein the memory comprises a non-volatile memory.

45. The implantable system of any one of the preceding claims, wherein the controller is further configured to adjust the opening pressure threshold of the valve based on one or more of a machine learning model, artificial intelligence, and signal processing techniques.

46. The implantable system of any one of the preceding claims, wherein the machine learning model is configured to determine the opening pressure threshold of the valve based on the ICP, the first measurement, the second measurement, the third measurement, the CSF flow rate, or any combination thereof, and wherein the controller is configured to adjust the opening pressure threshold of the valve.

47. The implantable system of any one of the preceding claims, wherein the status comprises one or more of a sleeping status, an awake status, an exercising status, a standing status, a sitting status, a laying status, and a circadian status.

48. The implantable system of any one of the preceding claims, wherein an algorithm is trained on a history of ICP waveforms, flow rate waveforms, or a combination thereof, and used to determine the status of the subject, wherein Fourier analysis or other signal processing techniques are applied to the ICP waveforms or flow rate waveforms, or both, to generate training data for the algorithm.

49. The implantable system of any one of the preceding claims, wherein the controller is configured to determine a rate of number of times the valve opens per unit of time, and wherein the rate is outputted, used to train the algorithm, or a combination thereof.

50. The implantable system of any one of the preceding claims, wherein the first pressure sensor comprises:
(a) a capacitive sensor;
(b) a piezoelectric sensor;
(c) a piezoresistive sensor;
(d) a magnetic sensor;
(e) a resonant sensor;
(f) an optical sensor;
(g) a MEMS sensor; or
(h) any combination thereof.

51. The implantable system of any one of the preceding claims, wherein the CSF flow rate sensor comprises:
(a) a thermal sensor;
(b) an optical sensor;
(c) an electromagnetic sensor;
(d) a Lorentz force velocity sensor;
(e) an ultrasonic sensor; or
(f) any combination thereof.

52. The implantable system of any one of the preceding claims, further comprising an energy storage device configured to power the first pressure sensor, the flow rate sensor, the controller, the accelerometer, or any combination thereof.

53. The implantable system of any one of the preceding claims, wherein the wireless charging circuit is configured to receive power to charge the energy storage device, power the controller, or both.

54. The implantable system of any one of the preceding claims, wherein the energy storage device comprises a battery, a capacitor, a supercapacitor, or any combination thereof.

55. The implantable system of any one of the preceding claims, wherein the controller is further configured to adjust the opening pressure threshold to a base opening pressure threshold if a charge of the energy storage device falls below a preset charge threshold.

56. The implantable system of any one of the preceding claims, further comprising a digital modulator configured to modulate a signal from the controller, and wherein the controller and the shunt wireless communication device are communicably coupled by the digital modulator.

57. The implantable system of any one of the preceding claims, further comprising a first analog to digital (A/D) converter communicably coupling the first pressure sensor and the controller and configured to convert an analog signal from the first pressure sensor to a digital signal.

58. The implantable system of any one of the preceding claims, further comprising an A/D converter communicably coupling the flow rate sensor and the controller and configured to convert an analog signal from the flow rate sensor to a digital signal.

59. The implantable system of any one of the preceding claims, wherein the controller comprises a digital modulator, one or more A/D converters, or any combination thereof.

60. The implantable system of any one of the preceding claims, wherein a P1 component and a P2 component of an ICP waveform are compared to determine if the subject's ICP is healthy or elevated, wherein an increased amplitude of the P2 component relative to the P1 component indicate that a subject has an elevated ICP.

61. The implantable system of any one of the preceding claims, wherein an alert is sent to the external device when a ratio of the P2 component to the P1 component of the ICP waveform exceeds a threshold indicating that the subject has an elevated ICP.

62. The implantable system of any one of the preceding claims, wherein an alert is sent to the external device if a difference between the P2 component and the P1 component of the ICP waveform exceeds a threshold indicating that the subject has an elevated ICP.

63. A platform for monitoring intracranial pressure (ICP) of cerebrospinal fluid (CSF) in a subject, the platform comprising:
(a) the implantable system of any of the preceding claims; and
(b) a wand device comprising a wand wireless communication device configured to transmit the atmospheric pressure and receive the ICP from a shunt wireless communication device.

64. The platform of any one of the preceding claims, wherein the first measurement includes the absolute ICP, atmospheric pressure, and sensor drift, and wherein the second measurement includes an atmospheric pressure and sensor drift, wherein the difference between the first and second measurements is the drift corrected, absolute ICP.

65. The platform of any one of the preceding claims, wherein the implantable system further comprises a motor configured to adjust an opening pressure threshold of the valve, wherein the wand wireless communication device is further configured to transmit a target opening pressure threshold to the implantable system, and wherein the controller is further configured to receive the target opening pressure threshold and control the motor to adjust the opening pressure threshold of the valve based at least in part on the target opening pressure threshold.

66. The platform of any one of the preceding claims, wherein an accelerometer is configured to output subject position data to the microcontroller, wherein upon detecting a transition between positions, the controller can direct the motor to reduce CSF flow and in so doing, act as an electronically controlled anti-siphon device (ASD).

67. The platform of any one of the preceding claims, wherein the controller is further configured to adjust the opening pressure threshold of the valve based at least in part on the ICP, the first measurement, the second measurement, or any combination thereof.

68. The platform of any one of the preceding claims, wherein the controller is further configured to receive a motor position from a motor position sensor, and wherein the shunt wireless communication device is further configured to transmit an opening pressure threshold confirmation based on the motor position.

69. The platform of any one of the preceding claims, wherein the wand wireless communication device is further configured to receive the opening pressure threshold confirmation.

70. The platform of any one of the preceding claims, wherein the controller is further configured to adjust the opening pressure threshold of the valve based on one or more of a machine learning model, artificial intelligence, or a signal processing algorithm.

71. The platform of any one of the preceding claims, wherein a system algorithm is configured to determine a status of the subject based on the ICP, the first measurement, the second measurement, or any combination thereof, and wherein the controller is configured to adjust the opening pressure threshold of the valve based on the status.

72. The platform of any one of the preceding claims, wherein the status comprises one or more of a sleeping status, an awake status, an exercising status, a standing status, a sitting status, a laying status, and a circadian status.

73. The platform of any one of the preceding claims, wherein a system algorithm is trained on a history of ICP waveforms, flow rate waveforms, or a combination thereof, and used to determine the status of the subject, wherein Fourier analysis or other signal processing techniques are be applied to the ICP waveforms, flow rate waveforms, or a combination thereof, to generate the training data for an algorithm.

74. The platform of any one of the preceding claims, wherein the controller is configured to determine a valve opening rate, and wherein the valve opening rate is outputted, used to train an algorithm, or a combination thereof.

75. The platform of any one of the preceding claims, wherein the implantable system further comprises a memory configured to store the first measurement, the second measurement, absolute ICP, ICP, the target ICP, or both.

76. The platform of any one of the preceding claims, wherein access to implantable system data, including ICP, absolute ICP, and flow rate, is restricted by a security code.

77. The platform of any one of the preceding claims, wherein a status of the shunt and subject is reported via a software application residing on the wand device, wherein the status comprises information including: shunt health, mean CSF flow rate, mean ICP, or any combination thereof.

78. The platform of any one of the preceding claims, wherein the memory comprises a non-volatile memory.

79. The platform of any one of the preceding claims, wherein the valve comprises one or more of a ball-spring valve, a floating ball valve, a trunnion mounted ball valve, a top entry ball valve, a side entry ball valve, a three-way ball valve, a ball check valve, a flapper check valve, a disc check valve, a ball relief valve, a spring safety valve, and a spring-loaded pressure relief valve.

80. The platform of any one of the preceding claims, wherein the first pressure sensor comprises:
(a) a capacitive sensor;
(b) a piezoelectric sensor;
(c) a piezoresistive sensor;
(d) a magnetic sensor;
(e) a resonant sensor;
(f) an optical sensor;
(g) a MEMS sensor; or
(h) any combination thereof.

81. The platform of any one of the preceding claims, wherein the implantable system further comprises an energy storage device configured to power the valve, the first pressure sensor, the second pressure sensor, the controller, or any combination thereof.

82. The platform of any one of the preceding claims, further comprising a wireless charging circuit configured to receive power to charge the energy storage device, power the controller, or a combination thereof.

83. The platform of any one of the preceding claims, wherein the energy storage device comprises a battery, a capacitor, a supercapacitor, or any combination thereof.

84. The platform of any one of the preceding claims, wherein the controller of the implantable system is configured to adjust the opening pressure threshold to a base opening pressure threshold if a charge of the energy storage device falls below a preset charge threshold.

85. The platform of any one of the preceding claims, further comprising a digital modulator, and wherein the controller and the shunt wireless communication device are communicably coupled by the digital modulator.

86. The platform of any one of the preceding claims, further comprising a first analog to digital (A/D) converter communicably coupling the first pressure sensor and the controller.

87. The platform of any one of the preceding claims, further comprising a second A/D converter communicably coupling the second pressure sensor and the controller.

88. The platform of any one of the preceding claims, wherein the controller comprises a digital modulator, one or more A/D converters, or any combination thereof.

89. The platform of any one of the preceding claims, further comprising a third A/D converter communicably coupling the flow rate sensor and the controller.

90. The platform of any one of the preceding claims, wherein the wand wireless communication device is further configured to transmit a target opening pressure, wherein the shunt wireless communication device is further configured to receive the target opening pressure threshold, and wherein the controller is configured to direct the motor based on the target opening pressure threshold.

91. The platform of any one of the preceding claims, wherein:
(a) the wand device further comprises a display configured to show data based on the first measurement, second measurement, the ICP, or both;
(b) the wand device further comprises a speaker configured to emit a sound based on the ICP, CSF flow rate, or both; or
(c) the wand wireless communication device is further configured to transmit the first measurement, second measurement, the third measurement, the ICP, CSF flow rate, or any combination thereof to a cloud storage.

92. The platform of any one of the preceding claims, wherein:
(a) the wand device further comprises a display configured to show data based on the opening pressure threshold confirmation;
(b) the wand device further comprises a speaker configured to emit a sound based on the opening pressure threshold confirmation;
(c) the wand wireless communication device is further configured to transmit the opening pressure threshold confirmation to a cloud storage; or
(d) any combination thereof.

93. The platform of any one of the preceding claims, wherein:
(a) the wand device further comprises a display configured to show data based on the target opening pressure;
(b) the wand wireless communication device is further configured to transmit the target opening pressure to a cloud storage; or
(c) both.

94. The platform of any one of the preceding claims, wherein:
(a) the wand device, typically a smartphone running an app, is capable of uploading patient data to a private or hybrid cloud accessible by the patients' doctors, and
(b) applications in the cloud can store the patient data and further process the patient data for doctors to make informed clinical diagnoses, wherein these data comprise mean ICP, mean CSF flow rate, valve opening rate, AIr, signal processing information from a system algorithm, or a combination thereof.

95. A computer-implemented method for monitoring intracranial pressure (ICP) of cerebrospinal fluid (CSF) in a subject, the method comprising:
(a) measuring, by an ICP sensor, an absolute ICP plus drift of the subject as a first measurement;
(b) measuring, by a second sensor, an atmospheric pressure plus drift as a second measurement;
(c) receiving the atmospheric pressure from an external source as the third measurement;
(d) calculating an absolute ICP based at least in part on a difference between the first and second measurements;
(e) calculating the ICP based at least in part on a difference between the absolute ICP and the third measurement; and
(f) transmitting the ICP.

96. The computer-implemented method of any one of the preceding claims, wherein the first measurement includes the absolute ICP, atmospheric pressure, and sensor drift, and wherein the second measurement includes an atmospheric pressure and the sensor drift.

97. The computer-implemented method of any one of the preceding claims, wherein the third measurement is from a smartphone.

98. The computer-implemented method of any one of the preceding claims, further comprising adjusting an opening pressure of a valve.

99. The computer-implemented method of any one of the preceding claims, wherein the adjustment is performed manually.

100. The computer-implemented method of any one of the preceding claims, wherein the adjustment is performed by a magnetic device.

101. The computer-implemented method of any one of the preceding claims, wherein the adjustment is performed by a motor.

102. The computer-implemented method of any one of the preceding claims, further comprising receiving a target opening pressure threshold, wherein the adjustment is performed based on the target opening pressure threshold.

103. The computer-implemented method of any one of the preceding claims, further comprising storing the target opening pressure threshold to a memory.

104. The computer-implemented method of any one of the preceding claims, further comprising storing the ICP, the first measurement, the second measurement, the third measurement, or any combination thereof to a memory.

105. The computer-implemented method of any one of the preceding claims, wherein the adjustment is performed based at least in part on the ICP, the first measurement, the second measurement, the third measurement, or any combination thereof.

106. The computer-implemented method of any one of the preceding claims, wherein the adjustment is performed based on an algorithm comprising one or more of a machine learning model, artificial intelligence model, a signal processing algorithm, or any combination thereof.

107. The computer-implemented method of any one of the preceding claims, wherein the algorithm is configured to determine a status of the subject based on the ICP, the first measurement, the second measurement, the third measurement, or any combination thereof.

108. The computer-implemented method of any one of the preceding claims, wherein the status of the subject comprises one or more of a sleeping status, an awake status, an exercising status, a standing status, a sitting status, a laying status, and a circadian status.

109. The computer-implemented method of any one of the preceding claims, wherein a system algorithm is trained on a history of ICP waveforms and used to determine the status of the subject, wherein Fourier analysis or other signal processing techniques are applied on the ICP waveforms to generate the training data for an algorithm.

110. The computer-implemented method of any one of the preceding claims, wherein the controller is configured to determine a rate of number of times the valve opens per unit of time, and wherein the rate is outputted, used to train an algorithm, or a combination thereof.

111. The computer-implemented method of any one of the preceding claims, further comprising adjusting the opening pressure threshold to a base opening pressure threshold if a charge of the energy storage device falls below a preset charge threshold.

112. Non-transitory computer-readable media comprising executable instructions that, when executed, cause at least one computer processor to perform the methods of any one of the preceding claims.

113. A computer system comprising a memory storing computer-readable instructions and at least one processor configured to execute the computer-readable instructions that are configured to perform the method of any of the preceding claims.
